(19) United States
(12) Patent Application Publication
(10) Pub. No.: US 2021/0181211 A1
(43) Pub. Date: Jun. 17, 2021

(54) CIRCULATING TFPI-2 (TISSUE FACTOR PATHWAY INHIBITOR 2) IN THE ASSESSMENT OF ATRIAL FIBRILLATION AND ANTICOAGULATION THERAPY

(71) Applicants: Roche Diagnostics Operations, Inc., Indianapolis, IN (US); Universiteit Maastricht, LK Maastricht (DE); Academisch Zickenhuis Maastricht, HX Maastricht (DE)

(72) Inventors: Peter Kastner, Penzberg (DE); Andre Ziegler, Rotkreuz (CH); Ursula-Henrike Wienhues-Thelen, Penzberg (DE); Vinzent Rolny, Penzberg (DE); Manuel Dietrich, Mannheim (DE); Ulrich Schotten, GT Maastricht (DE)

(21) Appl. No.: 17/176,797

(22) Filed: Feb. 16, 2021

Related U.S. Application Data
(63) Continuation of application No. PCT/EP2019/071999, filed on Aug. 16, 2019.

(30) Foreign Application Priority Data
Aug. 16, 2018 (EP) 18189328.0

(51) Int. Cl. G01N 33/68

(52) U.S. Cl. CPC G01N 33/6893; G01N 2496/00; G01N 2800/52; G01N 2800/2871; G01N 2800/50; G01N 2800/326

(57) ABSTRACT

The present invention relates to a method for assessing atrial fibrillation in a subject, said method comprising the steps of determining the amount of TFPI-2 in a sample from the subject, and comparing the amount of TFPI-2 to a reference amount, whereby atrial fibrillation is to be assessed. Moreover, the present invention relates to methods for assessing anticoagulation therapy and to methods for predicting the risk of stroke of a subject. Said methods are based on the determination of the amount of TFPI-2 in a sample from the subject.

The present invention relates to a method for assessing atrial fibrillation in a subject, said method comprising the steps of determining the amount of TFPI-2 in a sample from the subject, and comparing the amount of TFPI-2 to a reference amount, whereby atrial fibrillation is to be assessed. Moreover, the present invention relates to methods for assessing anticoagulation therapy and to methods for predicting the risk of stroke of a subject. Said methods are based on the determination of the amount of TFPI-2 in a sample from the subject.

### BACKGROUND SECTION

Atrial fibrillation (AF) is the most common type of heart arrhythmia and one of the most widespread conditions among the elderly population. Atrial fibrillation is characterized by irregular heart beating and often starts with brief periods of abnormal beating that can increase over time and may become a permanent condition. An estimated 2.7-6.1 million people in the United States have Atrial Fibrillation and approximately 33 million people globally (Chugh S. S. et al., Circulation 2014; 129:837-47).

The diagnosis of heart arrhythmia such as atrial fibrillation typically involves determination of the cause of the arrhythmia, and classification of the arrhythmia. Guidelines for the classification of atrial fibrillation according to the American College of Cardiology (ACC), the American Heart Association (AHA), and the European Society of Cardiology (ESC) are mainly based on simplicity and clinical relevance. The first category is called “first detected AF”. People in this category are initially diagnosed with AF and may or may not have had previous undetected episodes. If a first detected episode stops on its own in less than one week, but is followed by another episode later on, the category changes to “paroxysmal AF”. Although patients in this category have episodes lasting up to 7 days, in most cases of paroxysmal AF the episodes will stop in less than 24 hours. If the episode lasts for more than one week, it is classified as “persistent AF”. If such an episode cannot be stopped, i.e. by electrical or pharmacologic cardioversion, and continues for more than one year, the classification is changed to “permanent AF”.

An early diagnosis of atrial fibrillation is highly desired because atrial fibrillation is an important risk factor for stroke and systemic embolism (Hart et al., Ann Intern Med 2007; 146(12): 857-67; Go A S et al. JAMA 2001; 285(18): 2370-5). Stroke ranks after ischemic heart disease second as a cause of lost disability—adjusted—life years in high income countries and as a cause of death worldwide. In order to reduce the risk of stroke, anticoagulation therapy appears the most appropriate therapy.

TFPI-2 (tissue factor pathway inhibitor 2), also known as TFPI2, PP5, REF1, is a homologue of TFP-1 and contains three Kunitz-type domains and a basic C terminus region. It belongs to the Kunitz-type family which encompasses serine proteinase inhibitors that include one or more Kunitz-type domains of over twenty members, which includes bovine pancreatic trypsin inhibitor (aprotinin), tissue factor pathway inhibitor (TFPI-1) and its homolog, type-2 tissue factor pathway inhibitor, or TFPI-2. Human TFPI-2 was originally isolated from placental tissue as a 30-36 kDa glycoprotein which was referred to as placental protein 5 (PP5).

TFPI-2 is a serine proteinase inhibitor synthesized by a variety of cells and directionally secreted into their extracellular matrix (ECM) where it is thought to regulate plasmin-mediated ECM degradation and remodeling (reviewed by Chand et al.). The protein can inhibit a variety of serine proteases including factor VIIa/tissue factor, factor Xa, plasmin, trypsin, chymotryspin and plasma kallikrein, certain matrix metalloproteinases.

The TFPI-2 encoding gene has been also identified as a tumor suppressor gene in several types of cancer. TFPI-2 is downregulated in most aggressive tumors such as glioma, non-small cell lung cancer (NSCLC), breast cancer, melanoma, colorectal cancer, pancreatic cancer and hepatocellular carcinoma. Such silencing is mainly due to epigenetic changes induced by TFPI-2 promoter hypermethylation and histone deacetylation. TFPI-2 hypermethylation was detected e.g. for gastric/colorectal cancer (Hu et al. Oncotarget, 2017, Vol. 8, (No. 48), pp: 84054-84065).

TFPI-2 further plays a role in the regulation of coagulation and inhibits clot fibrinolysis. Vadivel et al. (J Biol Chem 2014; 289:31647-31661) show that platelets contain TFPI-2 derived from megakaryocytes and inhibits fibrinolysis. TFPI-2 regulates coagulation and tPA-induced fibrinolysis and promotes clot stabilization.

The role of TFPI-2 in atherosclerosis has been described e.g. by Crawley et al. (Arterioscler Thromb Vasc Biol. 2002; 22:218-224). In healthy human blood vessels TFPI-2 was detected in vascular endothelium. In human atherosclerotic tissues, TFPI-2 expression was assigned to macrophages, T cells, endothelial cells, and smooth muscle cells.

Furthermore, Herman et al. (J. Clin. Invest. 2001, 107:1117-1126) report that TFPI-2 function as a matrix metalloproteinase inhibitor with implications for arteriosclerosis.

Latini R. et al. (J Intern Med. 2011 February; 269(2): 160-71) measured various circulating biomarkers (hsTnT, NT-proBNP, MR-proANP, MR-proADM, copeptin, and CT-proendothelin-1) in patients with atrial fibrillation.

There is a need for reliable methods for the assessment of atrial fibrillation including the diagnosis of atrial fibrillation, the risk stratification of patients with atrial fibrillation (such as occurrence of stroke), the assessment of the severity of atrial fibrillation, and the assessment of a therapy in patients with atrial fibrillation.

The technical problem underlying the present invention can be seen as the provision of methods for complying with the aforementioned needs. The technical problem is solved by the embodiments characterized in the claims and herein below.

Advantageously, it was found in the context of the studies of the present invention that the determination of the amount of TFPI-2 in a sample from a subject allows for an improved assessment of atrial fibrillation. Thanks to present invention, it can be e.g. diagnosed whether a subject suffers from atrial fibrillation or not. The invention also provides a method for stroke prediction. Further, it can be e.g. differentiated between paroxysmal and persistent atrial fibrillation in a subject suffer from atrial fibrillation.

### BRIEF SUMMARY OF THE PRESENT INVENTION

The present invention relates to a method for assessing atrial fibrillation in a subject, comprising the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby atrial fibrillation is to be assessed.

The present invention further relates to a method for predicting the risk of stroke in a subject, comprising the steps of
(a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
(b) assessing the clinical stroke risk score for said subject, and
(c) predicting the risk of stroke based on the results of steps a) and b).

The present invention further relates to a method for improving the prediction accuracy of a clinical stroke risk score for a subject, comprising the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), wherein the subject has a known clinical risk stroke risk score, and
b) combining a value for the amount of TFPI-2 and/or the amount of one or more biomarkers comprising of a natriuretic peptide, ESM-1, ANGT2, IGFBP7 with the clinical stroke risk score, whereby the prediction accuracy of said clinical stroke risk score is improved.

The present invention further relates to a method for assessing the efficacy of an anticoagulation therapy of a subject, comprising the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
a) comparing the amount of the biomarker TFPI-2 to a reference amount for TFPI-2 and, optionally, comparing the amount of the at least one further biomarker to a reference amount for said at least one further biomarker, whereby the efficacy of the anticoagulation therapy is to be assessed.

The present invention further relates to a method for identifying a subject being eligible to the administration of at least one anticoagulation medicament or being eligible to an intensified anticoagulation therapy, comprising
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby a subject being eligible to the administration of said at least one medicament or to an intensified anticoagulation therapy is identified.

The present invention further relates to a method for monitoring anticoagulation therapy, comprising the steps of
(a) determining, in at first sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7),
(b) determining, in a second sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), wherein said second sample has been obtained after said first sample,
(c) comparing the amount of TFPI-2 in the first sample to the amount of TFPI-2 in said second sample, and optionally comparing the amount of said at least one further biomarker in the first sample to the amount of said at least one further biomarker in said second sample, thereby monitoring anticoagulation therapy.

The present invention further relates to a method of aiding in the assessment of atrial fibrillation, said method comprising the steps of:
a) providing at least one sample from a subject,
b) determining, in the at least one sample provided in step a), the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 and IGFBP7 (Insulin-like growth factor-binding protein 7), and
c) providing information on the determined amount of the biomarker TFPI-2 and optionally on the determined amount of the at least one further biomarker to a physician, thereby aiding in the assessment of atrial fibrillation.

The present invention further relates to a method for aiding in the assessment of atrial fibrillation, comprising:
a) providing an assay for the biomarker TFPI-2 and, optionally, at least one further assay for a further biomarker selected from the group consisting of a natriuretic peptide, ESM-1 (Endocan), Ang2 and IGFBP7 (Insulin-like growth factor-binding protein 7), and
b) providing instructions for using of assay results obtained or obtainable by said assay(s) in the assessment of atrial fibrillation.

The present invention further relates to a computer-implemented method for assessing atrial fibrillation, comprising
a) receiving, at a processing unit, a value for the amount of TFPI-2, and, optionally at least one further value for the amount of at least one further biomarker selected from the group consisting of a natriuretic peptide, ESM-1 (Endocan), Ang2 and IGFBP7 (Insulin-like growth factor-binding protein 7), wherein said amount of TFPI-2 and, optionally, the amount of the at least one further biomarker have been determined in a sample from a subject,
b) comparing, by said processing unit, the value or values received in step (a) to a reference or to references, and
c) assessing atrial fibrillation based in the comparison step b).

The present invention further relates to a kit comprising an agent which specifically binds to TFPI-2 and at least one further agent selected from the group consisting an agent which specifically binds to a natriuretic peptide, an agent which specifically binds to ESM-1, an agent which specifically binds Ang2 and an agent which specifically binds to IGFBP7.

The present invention further relates to the in vitro use of
i) the biomarker TFPI-2 and optionally of at least one further biomarker selected from the group consisting of a natriuretic peptide, ESM-1 (Endocan), Ang2 and IGFBP7 (Insulin-like growth factor-binding protein 7), and/or
ii) at least one agent that specifically binds to TFPI-2, and, optionally, at least one further agent selected from the group consisting an agent which specifically binds to a natriuretic peptide, an agent which specifically binds to ESM-1, an agent which specifically binds to Ang2 and an agent which specifically binds to IGFBP7,
   for a) assessing atrial fibrillation, b) predicting the risk of stroke in a subject, c) improving the prediction accuracy of a clinical stroke risk score, for assessing the efficacy of an anticoagulation therapy of a subject, d) for identifying a subject being eligible to the administration of at least one anticoagulation medicament or being eligible to an intensified anticoagulation therapy, and/or e) monitoring anticoagulation therapy.

In an embodiment, the assessment of atrial fibrillation is the diagnosis of atrial fibrillation.

In an embodiment, an amount of TFPI-2 and, optionally, an amount of the at least one further biomarker above the reference amount is indicative for a subject suffering from atrial fibrillation and/or an amount of TFPI-2 and, optionally, an amount of the at least one further biomarker below the reference amount is indicative for a subject not suffering from atrial fibrillation.

In an embodiment of the present invention, the assessment of atrial fibrillation is the differentiation between paroxysmal and persistent atrial fibrillation.

In an embodiment of the present invention, an amount of TFPI-2 and, optionally, an amount of the at least one further biomarker above the reference amount is indicative for a subject suffering from persistent atrial fibrillation and/or wherein an amount of TFPI-2 and, optionally, an amount of the at least one further biomarker below the reference amount is indicative for a subject suffering from paroxysmal atrial fibrillation.

In an embodiment of the present invention, the assessment of atrial fibrillation is the prediction of the risk of an adverse event associated with atrial fibrillation.

In an embodiment of the present invention, the adverse event associated with atrial fibrillation is recurrence of atrial fibrillation and/or stroke.

In an embodiment of the present invention, an amount of TFPI-2 and, optionally, an amount of the at least one further biomarker above the reference amount is indicative for a subject who is at risk of suffering from an adverse event associated with atrial fibrillation and/or an amount of TFPI-2 and, optionally, an amount of the at least one further biomarker below the reference amount is indicative for a subject who is not at risk of suffering from an adverse event associated with atrial fibrillation.

In an embodiment of the present invention, the assessment of atrial fibrillation is the identification of a subject who shall be subjected to electrocardiography (ECG).

### DETAILED OVERVIEW ON THE PRESENT INVENTION AND DEFINITIONS

The present invention relates to a method for assessing atrial fibrillation in a subject, comprising the steps of
a) determining, in at least one sample from the subject, the amount of the biomarker TFPI-2 (Tissue Factor Pathway Inhibitor 2) and
b) comparing the amount of the biomarker TFPI-2 to a reference amount for TFPI-2, whereby atrial fibrillation is to be assessed.

In an embodiment of method of the present invention, the method further comprises the determination of the amount of at least one further biomarker selected from the group consisting of a natriuretic peptide, ESM-1 (Endocan), Ang2 (Angiopoietin 2) and IGFBP7 (Insulin-like growth factor-binding protein 7) in a sample from the subject in step a) and the comparison of the amount of the at least one further biomarker to a reference amount in step b).

Accordingly, the present invention relates to a method for assessing atrial fibrillation in a subject, comprising the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby atrial fibrillation is to be assessed.

The assessment of atrial fibrillation (AF) shall be based on the results of the comparison step b).

Accordingly, the present invention preferably comprises the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7),
b) comparing the amount of the biomarker TFPI-2 to a reference amount for TFPI-2 and, optionally, comparing the amount of the at least one further biomarker to a reference amount for said at least one further biomarker, and
c) assessing atrial fibrillation based on the results of the comparison step b).

The method as referred to in accordance with the present invention includes a method which essentially consists of the aforementioned steps or a method which includes further steps. Moreover, the method of the present invention, preferably, is an ex vivo and more preferably an in vitro method. Moreover, it may comprise steps in addition to those explicitly mentioned above. For example, further steps may relate to the determination of further markers and/or to sample pre-treatments or evaluation of the results obtained by the method. The method may be carried out manually or assisted by automation. Preferably, step (a), (b) and/or (c) may in total or in part be assisted by automation, e.g., by a suitable robotic and sensory equipment for the determination in step (a) or a computer-implemented calculation in step (b).

In accordance with the present invention, atrial fibrillation shall be assessed. The term “assessing atrial fibrillation” as used herein preferably refers to the diagnosis of atrial fibrillation, the differentiation between paroxysmal and persistent atrial fibrillation, the prediction of a risk of an adverse event associated with atrial fibrillation (such as stroke), to the identification of a subject who shall be subjected to electrocardiography (ECG), or to the assessment of a therapy for atrial fibrillation.

As will be understood by those skilled in the art, the assessment of the present invention is usually not intended to be correct for 100% of the subjects to be tested. The term, preferably, requires that a correct assessment (such as the diagnosis, differentiation, prediction, identification or assessment of a therapy as referred to herein) can be made for a statistically significant portion of subjects. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.4, 0.1, 0.05, 0.01, 0.005, or 0.0001.

It is known in the art that biomarkers could be increased in various diseases and disorders. This does also apply to TFPI-2 which is e.g. known to be increased in cancer patients. However, this is taken into account by the skilled person.

Accordingly, the expression “assessment of atrial fibrillation” is understood as an aid in the assessment of atrial fibrillation, and thus as an aid in diagnosing atrial fibrillation, an aid in differentiating between paroxysmal and persistent atrial fibrillation, an aid in the prediction of a risk of an adverse event associated with atrial fibrillation, an aid in the identification of a subject who shall be subjected to electrocardiography (ECG), or as an aid in the assessment of a therapy for atrial fibrillation. The final diagnosis, in principle, will be carried out by physician.

In a preferred embodiment of the present invention, the assessment of atrial fibrillation is the diagnosis of atrial fibrillation. Accordingly, it is diagnosed, whether a subject suffers from atrial fibrillation, or not.

Accordingly, the present invention envisages a method for diagnosing atrial fibrillation in a subject, comprising the steps of
a) determining the amount of TFPI-2 in a sample from the subject, and
b) comparing the amount of TFPI-2 to a reference amount, whereby atrial fibrillation is to be diagnosed.

In an embodiment, the aforementioned method comprises the steps of:
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby atrial fibrillation is to be diagnosed.

Preferably, the subject to be tested in connection with method for diagnosing of atrial fibrillation is a subject who is suspected to suffer from atrial fibrillation. However, it is also contemplated that the subject already has been diagnosed previously to suffer from AF and that the previous diagnosis is confirmed by carrying out the method of the present invention.

In another preferred embodiment of the present invention, the assessment of atrial fibrillation is the differentiation between paroxysmal and persistent atrial fibrillation. Accordingly, it is determined whether a subject suffers from the paroxysmal or persistent atrial fibrillation.

Accordingly, the present invention envisages a method for differentiating between paroxysmal and persistent atrial fibrillation in a subject, comprising the steps of
a) determining the amount of TFPI-2 in a sample from the subject, and
b) comparing the amount of TFPI-2 to a reference amount, whereby it is differentiated between paroxysmal and persistent atrial fibrillation.

In an embodiment, the aforementioned method comprises the steps of:
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby it is differentiated between paroxysmal and persistent atrial fibrillation.

In another preferred embodiment of the present invention, the assessment of atrial fibrillation is the prediction of the risk of an adverse event associated with atrial fibrillation (such as stroke). Accordingly, it is predicted whether a subject is at risk and/or not as risk of said adverse event.

Thus, the present invention envisages a method for predicting the risk of an adverse event associated with atrial fibrillation in a subject, comprising the steps of
a) determining the amount of TFPI-2 in a sample from the subject, and
b) comparing the amount of TFPI-2 to a reference amount, whereby the risk of the adverse event associated with atrial fibrillation is to be predicted.

In an embodiment, the aforementioned method comprises the steps of:
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby the risk of the adverse event associated with atrial fibrillation is to be predicted.

It is envisaged that various adverse events can be predicted. A preferred adverse event to be predicted is stroke.

Accordingly, the present invention, in particular, contemplates a method for predicting the risk of stroke in a subject, comprising the steps of
a) determining the amount of TFPI-2 in a sample from the subject, and
b) comparing the amount of TFPI-2 to a reference amount, whereby the risk of stroke is to be predicted.

The aforementioned method may further comprise step c) of predicting stroke based on the comparison results of step b). Thus, steps a), b), c) are preferably as follows:
a) determining the amount of TFPI-2 in a sample from the subject, and
b) comparing the amount of TFPI-2 to a reference amount, and
c) predicting stroke based on the comparison results of step b)

In another preferred embodiment of the present invention, the assessment of atrial fibrillation is the assessment of a therapy for atrial fibrillation.

Accordingly, the present invention envisages a method for the assessment of a therapy for atrial fibrillation in a subject, comprising the steps of
a) determining the amount of TFPI-2 in a sample from the subject, and
b) comparing the amount of TFPI-2 to a reference amount, whereby the therapy for atrial fibrillation is to be assessed.

In an embodiment, the aforementioned method comprises the steps of:
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby the therapy for atrial fibrillation is to be assessed.

Preferably, the subject in connection with the aforementioned differentiation, the aforementinned prediction, and the assessment of a therapy for atrial fibrillation is a subject who suffers from atrial fibrillation, in particular who is known to suffer from atrial fibrillation (and thus to have a known history of atrial fibrillation). However, with respect to the aforementioned prediction method, it is also envisaged that the subject has no known history of atrial fibrillation.

In another preferred embodiment of the present invention, the assessment of atrial fibrillation is the identification of a subject who shall be subjected to electrocardiography (ECG). Accordingly, a subject is identified who is who shall be subjected to electrocardiography, or not.

Thus, the present invention envisages a method for identifying a subject who shall be subjected to electrocardiography, comprising the steps of
a) determining the amount of TFPI-2 in a sample from the subject, and
b) comparing the amount of TFPI-2 to a reference amount, whereby a subject is identified who shall be subjected to electrocardiography.

In an embodiment, the aforementioned method comprises the steps of:
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby a subject is identified who shall be subjected to electrocardiography.

Preferably, the subject in connection with the aforementioned method of identifying a subject who shall be subjected to electrocardiography is a subject who has no known history of atrial fibrillation. The expression “no known history of atrial fibrillation” is defined elsewhere herein.

In another preferred embodiment of the present invention, the assessment of atrial fibrillation is the assessment of efficacy of an anticoagulation therapy of a subject. Accordingly, the efficacy of said therapy is assessed. The subject in accordance with this method may or may not suffer from atrial fibrillation.

In another preferred embodiment of the present invention, the assessment of atrial fibrillation is the monitoring of an anticoagulation therapy of a subject. The subject in accordance with this method may or may not suffer from atrial fibrillation.

In another preferred embodiment of the present invention, the assessment of atrial fibrillation is the prediction of the risk of stroke in a subject. Accordingly, it is predicted whether a subject as referred to herein is at risk of stroke, or not.

In another preferred embodiment of the present invention, the assessment of atrial fibrillation is the identification a subject being eligible to the administration of at least one anticoagulation medicament or being eligible to an intensified anticoagulation therapy. Accordingly, it is assessed whether a subject is eligible to said administration and/or said increase of the dosage.

In another preferred embodiment of the present invention, the assessment of atrial fibrillation is the monitoring of anticoagulation therapy. Accordingly, it is assessed whether a subject responds to said therapy, or not.

The term “atrial fibrillation” (“abbreviated” AF or AFib) is well known in the art. As used herein, the term preferably refers to a supraventricular tachyarrhythmia characterized by uncoordinated atrial activation with consequent deterioration of atrial mechanical function. In particular, the term refers to an abnormal heart rhythm characterized by rapid and irregular beating. It involves the two upper chambers of the heart. In a normal heart rhythm, the impulse generated by the sino-atrial node spreads through the heart and causes contraction of the heart muscle and pumping of blood. In atrial fibrillation, the regular electrical impulses of the sino-atrial node are replaced by disorganized, rapid electrical impulses which result in irregular heart beats. Symptoms of atrial fibrillation are heart palpitations, fainting, shortness of breath, or chest pain. However, most episodes have no symptoms. On the electrocardiogram, Atrial Fibrillation is characterized by the replacement of consistent P waves by rapid oscillations or fibrillatory waves that vary in amplitude, shape, and timing, associated with an irregular, frequently rapid ventricular response when atrioventricular conduction is intact.

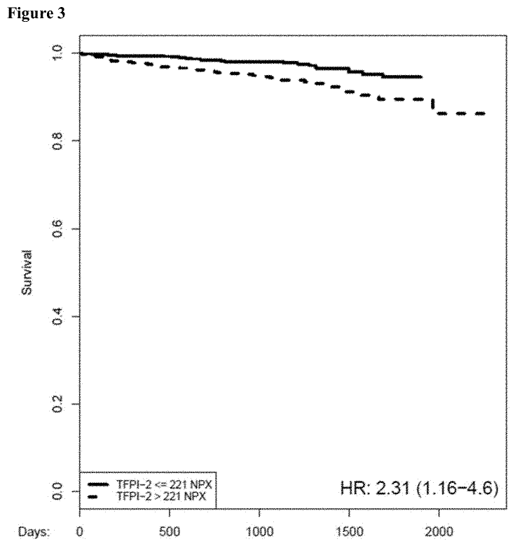

The American College of Cardiology (ACC), American Heart Association (AHA), and the European Society of Cardiology (ESC) propose the following classification system (see Fuster V. et al., Circulation 2006; 114 (7): e257-354 which herewith is incorporated by reference in its entirety, see e.g. FIG. 3 in the document): First detected AF, paroxysmal AF, persistent AF, and permanent AF.

All people with AF are initially in the category called first detected AF. However, the subject may or may not have had previous undetected episodes. A subject suffers from permanent AF, if the AF has persisted for more than one year, and in particular, conversion back to sinus rhythm does not occur (or only with medical intervention). A subject suffers from persistent AF, if the AF lasts more than 7 days. The subject may require either pharmacologic or electrical intervention to terminate Atrial Fibrillation. Preferably, persistent AF occurs in episodes, but the arrhythmia does not convert back to sinus rhythm spontaneously (i.e. without medical intervention). Paroxysmal Atrial Fibrillation, preferably, refers to an intermittent episode of Atrial Fibrillation which lasts up to 7 days. In most cases of paroxysmal AF, the episodes last less than 24 hours. The episode of Atrial Fibrillation terminates spontaneously, i.e. without medical intervention. Thus, whereas the episode(s) of paroxysmal atrial fibrillation preferably terminate spontaneously, persistent atrial fibrillation preferably does not end spontaneously. Preferably, persistent atrial fibrillation requires electrical or pharmacological cardioversion for termination, or other procedures, such as ablation procedures (Fuster V. et al., Circulation 2006; 114 (7): e257-354). Both persistent and paroxysmal AF may be recurrent, whereby distinction of paroxysmal and persistent AF is provided by ECG recordings: When a patient has had 2 or more episodes, AF is considered recurrent. If the arrhythmia terminates spontaneously, AF, in particular recurrent AF, is designated paroxysmal. AF is designated persistent if it lasts more than 7 days.

In a preferred embodiment of the present invention, the term “paroxysmal atrial fibrillation” is defined as episodes of AF that terminate spontaneously, wherein said episodes last less than 24 hours. In an alternative embodiment, the episodes which terminate spontaneously last up to seven days.

The “subject” as referred to herein is, preferably, a mammal Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human subject.

Preferably, the subject to be tested is of any age, more preferably, the subject to be tested is 50 years of age or older, more preferably 60 years of age or older, and most preferably 65 years of age or older. Further, it is envisaged that the subject to be tested is 70 years of age or older.

Moreover, it is envisaged that the subject to be tested is 75 years of age or older. Also, the subject may be between 50 and 90 years.

In a preferred embodiment of the method of assessing atrial fibrillation, the subject to be tested shall suffer from atrial fibrillation. Accordingly, the subject shall have a known history of atrial fibrillation. Thus, the subject shall have experienced episodes of Atrial Fibrillation prior to obtaining the test sample, and at least one of the previous episodes of atrial fibrillation shall have been diagnosed, e.g. by ECG. For example, it is envisaged that the subject suffers from atrial fibrillation, if the assessment of atrial fibrillation is the differentiation between paroxysmal and persistent atrial fibrillation, or if the assessment of atrial fibrillation is the prediction of a risk of an adverse event associated with atrial fibrillation, or if the assessment of atrial fibrillation is the assessment of a therapy for atrial fibrillation.

In another preferred embodiment of the method of assessing atrial fibrillation, the subject to be tested is suspected to suffer from atrial fibrillation, e.g. if the assessment of atrial fibrillation is the diagnosis of atrial fibrillation or the identification of a subject who shall be subjected to electrocardiography (ECG).

Preferably, a subject who is suspected to suffer from atrial fibrillation is a subject who has shown at least one symptom of atrial fibrillation prior to carrying out the method for assessing atrial fibrillation. Said symptoms are usually transient and may arise in a few seconds and may disappear just as quickly. Symptoms of atrial fibrillation include dizziness, fainting, shortness of breath and, in particular, heart palpitations. Preferably, the subject has shown at least one symptom of atrial fibrillation within six months prior to obtaining the sample.

Alternatively or additionally, a subject who is suspected to suffer from atrial fibrillation shall be a subject who is 70 years of age or older.

Preferably, the subject who is suspected to suffer from atrial fibrillation shall have no known history of atrial fibrillation.

In accordance with the present invention, a subject having no known history of atrial fibrillation is, preferably, a subject who has not been diagnosed to suffer from atrial fibrillation previously, i.e. before carrying out the method of the present invention (in particular before obtaining the sample from the subject). However, the subject may or may not have had previous undiagnosed episodes of atrial fibrillation.

Preferably, the term “atrial fibrillation” refers to all types of atrial fibrillation. Accordingly, the term preferably encompasses paroxysmal, persistent or permanent atrial fibrillation.

In an embodiment of the present invention, however, the subject to be tested does not suffer from permanent atrial fibrillation. In this embodiment, the term “atrial fibrillation” only refers to paroxysmal and persistent atrial fibrillation.

In another embodiment of the present invention, however, the subject to be tested does not suffer from paroxysmal and permanent atrial fibrillation. In this embodiment, the term “atrial fibrillation” only refers to persistent atrial fibrillation.

As set forth above, the biomarker TFPI-2 could be increased in various diseases and disorders other than atrial fibrillation. In an embodiment of the present invention, it is envisaged that the subject does not suffer from such diseases and disorders. For example, it is envisaged that the subject shall not suffer cancer and/or pancreatitis. The subject is not pregnant.

The subject to be tested may or may not experience episodes of atrial fibrillation when the sample is obtained. Thus, in a preferred embodiment of the assessment of atrial fibrillation (such as in the diagnosis of atrial fibrillation), the subject does not experience episodes of Atrial Fibrillation when the sample is obtained. In this embodiment, the subject shall have a normal sinus rhythm when the sample is obtained (and shall be accordingly in sinus rhythm). Thus, the diagnosis of atrial fibrillation is possible even in the (temporary) absence of atrial fibrillation. In accordance with the method of the present invention, the elevation of the biomarkers as referred to herein should be preserved after the episode of Atrial Fibrillation and, thus, provide a diagnosis of a subject who has suffered from Atrial Fibrillation. Preferably, the diagnosis of AF within about three days, within about one month, within about three months, or within about 6 months after carrying out the method of the present invention (or to be more precise after the sample has been obtained). In a preferred embodiment, the diagnosis of Atrial Fibrillation within about six months after the episode is feasible. In a preferred embodiment, the diagnosis of Atrial Fibrillation within about six months after the episode is feasible. Accordingly, the assessment of atrial fibrillation as referred to herein, in particular the diagnosis, the prediction of the risk or the differentiation as referred to herein in connection with the assessment of atrial fibrillation is preferably carried out after about three days, more preferably after about one month, even more preferably after about three month, and most preferably after about six months after the last episode of atrial fibrillation.

Consequently, is envisaged that is sample to be tested is preferably obtained after about three days, more preferably after about one month, even more preferably after about three month, and most preferably after about six months after the last episode of atrial fibrillation. Accordingly, the diagnosis of atrial fibrillation preferably also encompasses the diagnosis of episodes of atrial fibrillation that occurred preferably within about three days, more preferably within about three months, and most preferably within about six months before the sample was obtained.

However, it is also envisaged that the subject experiences episodes of atrial fibrillation when the sample is obtained (e.g. with respect to the prediction of stroke).

The method of the present invention can be also used for the screening of larger populations of subjects. Therefore, it is envisaged, that at least 100 subjects, in particular at least 1000 subjects are assessed with respect to atrial fibrillation. Thus, the amount of the biomarker is determined in samples from at least 100, or in particular of from at least 1000 subjects. Moreover, it is envisaged that at least 10,000 subjects are assessed.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the biomarker(s) in the sample.

In a preferred embodiment of the present invention, the sample is a blood (i.e. whole blood), serum or plasma sample. Serum is the liquid fraction of whole blood that is obtained after the blood is allowed to clot. For obtaining the serum, the clot is removed by centrifugation and the supernatant is collected. Plasma is the acellular fluid portion of blood. For obtaining a plasma sample, whole blood is collected in anticoagulant-treated tubes (e.g. citrate-treated or EDTA-treated tubes). Cells are removed from the sample by centrifugation and the supernatant (i.e. the plasma sample) is obtained.

As set forth above, the subject may be in sinus rhythm or may suffer from an episode of AF rhythm at the time at which the sample is obtained.

TFPI-2 (Tissue factor pathway inhibitor-2) is well known in the art. The protein is a member of the Kunitz-type serine proteinase inhibitor family. The protein can inhibit a variety of serine proteases including factor VIIa/tissue factor, factor Xa, plasmin, trypsin, chymotryspin and plasma kallikrein.

The biomarker TFPI-2 gene is located on human chromosome 7q22, and is a 32 kDa matrix-associated Kunitz-type serine proteinase inhibitor consisting of a short amino-terminal region, three tandem Kunitz-type domains and a positively charged carboxy-terminal tail. The protein can inhibit a variety of serine proteases including factor VIIa/tissue factor, factor Xa, plasmin, trypsin, chymotryspin and plasma kallikrein. This gene has been identified as a tumor suppressor gene in several types of cancer.

In a preferred embodiment of the present invention, the amount of the human TFPI-2 polypeptide is determined in a sample from the subject. The sequence of the human TFPI-2 polypeptide is well known in the art and can be e.g. assessed via Uniprot database, see UniProtKB—P48307 (TFPI2_HUMAN).

In humans, TFPI-2 gene maps to chromosome 7 at the 7q22 region. The human TFPI-2 gene spans 8164 nucleotide bases and consists of five exons and four introns. Each Kunitz-type domain is encoded by a single exon. The human TFPI-2 gene does not contain additional exons in the 5′-flanking region, or exons encoding the connecting regions present between the Kunitz-type domains. While TFPI-2 has one transcription termination site approximately 390 bp downstream (+4321) of the termination codon (TAA), there are three transcription initiation sites located 75 bp (13), 56 bp (11), and 38 bp upstream of the translation start site (ATG).

Alternative splicing results in multiple transcript variants of TFPI-2. This gene has 3 transcripts (splice variants), 91 orthologues and 8 paralogues.

In a preferred embodiment, the amount of isoform 1 of the TFPI-2 transcript is determined, i.e. isoform 1 having a sequence as shown under accession number ENST00000222543.9.

In another preferred embodiment, the amount of isoform 2 of the TFPI-2 transcript is determined, i.e. isoform 2 having a sequence as shown under accession number ENST00000451238.1.

In another preferred embodiment, the amount of isoform 3 of the TFPI-2 transcript is determined, i.e. isoform 3 having a sequence as shown under accession number ENST00000461482.1 (http://www.ensembl.org).

In another preferred embodiment, the amount of isoform-1, 2 and 3 of the TFPI-2 transcript is determined, i.e. total TFPI-2.

The term “natriuretic peptide” comprises atrial natriuretic peptide (ANP)-type and brain natriuretic peptide (BNP)-type peptides. Thus, natriuretic peptides according to the present invention comprise ANP-type and BNP-type peptides and variants thereof (see, e.g., Bonow R O. et al., Circulation 1996; 93: 1946-1950).

ANP-type peptides comprise pre-proANP, proANP, NT-proANP, and ANP.

BNP-type peptides comprise pre-proBNP, proBNP, NT-proBNP, and BNP.

The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NTpro peptide, 76 amino acids in case of NT-proBNP) and the active hormone (32 amino acids in the case of BNP, 28 amino acids in the case of ANP).

Preferred natriuretic peptides according to the present invention are NT-proANP, ANP, NT-proBNP, BNP. ANP and BNP are the active hormones and have a shorter half-life than their respective inactive counterparts, NT-proANP and NT-proBNP. BNP is metabolized in the blood, whereas NT-proBNP circulates in the blood as an intact molecule and as such is eliminated renally.

The most preferred natriuretic peptides according to the present invention are NT-proBNP and BNP, in particular NT-proBNP. As briefly discussed above, the human NT-proBNP as referred to in accordance with the present invention is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913, and Bonow R O. Et al., New Insights into the cardiac natriuretic peptides. Circulation 1996; 93: 1946-1950. Preferably, human NT-proBNP as used herein is human NT-proBNP as disclosed in EP 0 648 228 B1.

IGFBP-7 (Insulin-like Growth Factor Binding Protein 7) is a 30-kDa modular glycoprotein known to be secreted by endothelial cells, vascular smooth muscle cells, fibroblasts, and epithelial cells (Ono, Y., et al., Biochem Biophys Res Comm 202 (1994) 1490-1496). Preferably, the term “IGFBP-7” refers to human IGFBP-7. The sequence of the protein is well-known in the art and is e.g. accessible via UniProt (Q16270, IBP7_HUMAN), or via GenBank (NP_001240764.1). A detailed definition of the biomarker IGFBP-7 is e.g. provided in WO 2008/089994 which herewith is incorporated by reference in its entirety. There are two isoforms of IGFBP-7, Isoform 1 and 2 which are produced by alternative splicing. In an embodiment of the present invention, the total amount of both isoforms is measured (for the sequence, see the UniProt database entry (Q16270-1 and Q16270-2).

The biomarker endothelial cell specific molecule 1 (abbreviated ESM-1) is well known in the art. The biomarker is frequently also referred to as endocan. ESM-1 is a secreted protein which is mainly expressed in the endothelial cells in human lung and kidney tissues. Public domain data suggest expression also in thyroid, lung and kidney, but also in heart tissue, see. e.g. the entry for ESM-1 in the Protein Atlas database (Uhlén M. et al., Science 2015; 347(6220): 1260419). The expression of this gene is regulated by cytokines. ESM-1 is a proteoglycan composed of a 20 kDa mature polypeptide and a 30 kDa O-linked glycan chain (Bechard D et al., J Biol Chem 2001; 276(51):48341-48349).

In a preferred embodiment of the present invention, the amount of the human ESM-1 polypeptide is determined in a sample from the subject. The sequence of the human ESM-1 polypeptide is well known in the art (see e.g. Lassale P. et al., J. Biol. Chem. 1996; 271:20458-20464 and can be e.g. assessed via Uniprot database, see entry Q9NQ30 (ESM1_HUMAN). Two isoforms of ESM-1 are produced by alternative splicing, isoform 1 (having the Uniprot identifier Q9NQ30-1) and isoform 2 (having the Uniprot identifier Q9NQ30-2). Isoform 1 has length of 184 amino acids. In isoform 2, amino acids 101 to 150 of isoform 1 are missing. Amino acids 1 to 19 form the signal peptide (which might be cleaved off).

In a preferred embodiment, the amount of isoform 1 of the ESM-1 polypeptide is determined, i.e. isoform 1 having a sequence as shown under UniProt accession number Q9NQ30-1.

In another preferred embodiment, the amount of isoform 2 of the ESM-1 polypeptide is determined, i.e. isoform 2 having a sequence as shown under UniProt accession number Q9NQ30-2.

In another preferred embodiment, the amount of isoform-1 and isoform 2 of the ESM-1 polypeptide is determined, i.e. total ESM-1.

For example, the amount of ESM-1 could be determined with a monoclonal antibody (such as a mouse antibody) against amino acids 85 to 184 of the ESM-1 polypeptide and/or with a goat polyclonal antibody.

The biomarker Angiopoietin-2 (abbreviated “Ang-2”, frequently also referred to as ANGPT2) is well known in the art. It is a naturally occurring antagonist for both Ang-1 and TIE2 (see e.g. Maisonpierre et al., Science 277 (1997) 55-60). The protein can induce tyrosine phosphorylation of TEK/TIE2 in the absence of ANG-1. In the absence of angiogenic inducers, such as VEGF, ANG2-mediated loosening of cell-matrix contacts may induce endothelial cell apoptosis with consequent vascular regression. In concert with VEGF, it may facilitate endothelial cell migration and proliferation, thus serving as a permissive angiogenic signal. The sequence of human Angiopoietin is well known in the art. Uniprot lists three isoforms of Angiopoietin-2: Isoform 1 (Uniprot identifier: O15123-1), Isoform 2 (identifier: O15123-2) and Isoform 3 (O15123-3). In a preferred embodiment, the total amount of Angiopoietin-2 is determined. The total amount is preferably the sum of the amounts of complexed and free Angiopoietin-2.

The term “determining” the amount of a biomarker as referred to herein (such as TFPI-2 or the natriuretic peptide) refers to the quantification of the biomarker, e.g. to measuring the level of the biomarker in the sample, employing appropriate methods of detection described elsewhere herein. The terms “measuring” and “determining” are used herein interchangeably.

In an embodiment, the amount of a biomarker is determined by contacting the sample with an agent that specifically binds to the biomarker, thereby forming a complex between the agent and said biomarker, detecting the amount of complex formed, and thereby measuring the amount of said biomarker.

The biomarkers as referred to herein (such as TFPI-2) can be detected using methods generally known in the art. Methods of detection generally encompass methods to quantify the amount of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays and proximity extension assays, which are commercially available. Further suitable methods to detect biomarkers include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

For the detection of biomarker proteins as referred to herein a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Methods employing electrochemiluminescent labels are well-known. Such methods make use of the ability of special metal complexes to achieve, by means of oxidation, an excited state from which they decay to ground state, emitting electrochemiluminescence. For review see Richter, M. M., Chem. Rev. 2004; 104: 3003-3036.

In an embodiment, the detection antibody (or an antigen-binding fragment thereof) to be used for measuring the amount of a biomarker is ruthenylated or iridinylated. Accordingly, the antibody (or an antigen-binding fragment thereof) shall comprise a ruthenium label. In an embodiment, said ruthenium label is a bipyridine-ruthenium(II) complex. Or the antibody (or an antigen-binding fragment thereof) shall comprise an iridium label. In an embodiment, said iridium label is a complex as disclosed in WO 2012/107419.

In an embodiment of the sandwich assay for the determination of TFPI-2, the assay comprises a biotinylated first monoclonal antibody that specifically binds TFPI-2 (as capture antibody) and a ruthenylated F(ab′)2-fragment of a second monoclonal antibody that specifically binds TFPI-2 as detection antibody). The two antibodies form sandwich immunoassay complexes with TFPI-2 in the sample.

In an embodiment of the sandwich assay for the determination of the natriuretic peptide, the assay comprises a biotinylated first monoclonal antibody that specifically binds the natriuretic peptide (as capture antibody) and a ruthenylated F(ab′)2-fragment of a second monoclonal antibody that specifically binds the natriuretic peptide as detection antibody). The two antibodies form sandwich immunoassay complexes with the natriuretic peptide in the sample.

Measuring the amount of a polypeptide (such as TFPI-2 or the natriuretic peptide) may, preferably, comprise the steps of (a) contacting the polypeptide with an agent that specifically binds said polypeptide, (b) (optionally) removing non-bound agent, (c) measuring the amount of bound binding agent, i.e. the complex of the agent formed in step (a). According to a preferred embodiment, said steps of contacting, removing and measuring may be performed by an analyzer unit. According to some embodiments, said steps may be performed by a single analyzer unit of said system or by more than one analyzer unit in operable communication with each other. For example, according to a specific embodiment, said system disclosed herein may include a first analyzer unit for performing said steps of contacting and removing and a second analyzer unit, operably connected to said first analyzer unit by a transport unit (for example, a robotic arm), which performs said step of measuring.

The agent which specifically binds the biomarker (herein also referred to as “binding agent”) may be coupled covalently or non-covalently to a label allowing detection and measurement of the bound agent. Labeling may be done by direct or indirect methods. Direct labeling involves coupling of the label directly (covalently or non-covalently) to the binding agent. Indirect labeling involves binding (covalently or non-covalently) of a secondary binding agent to the first binding agent. The secondary binding agent should specifically bind to the first binding agent. Said secondary binding agent may be coupled with a suitable label and/or be the target (receptor) of a tertiary binding agent binding to the secondary binding agent. Suitable secondary and higher order binding agents may include antibodies, secondary antibodies, and the well-known streptavidin-biotin system (Vector Laboratories, Inc.). The binding agent or substrate may also be “tagged” with one or more tags as known in the art. Such tags may then be targets for higher order binding agents. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium complexes, iridium complexes, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, avail-able as ready-made stock solution from Roche Diagnostics), CDP-Star™ (Amersham Bio-sciences), ECF™ (Amersham Biosciences). A suitable enzyme-substrate combination may result in a colored reaction product, fluorescence or chemoluminescence, which can be determined according to methods known in the art (e.g. using a light-sensitive film or a suit-able camera system). As for measuring the enzymatic reaction, the criteria given above apply analogously. Typical fluorescent labels include fluorescent proteins (such as GFP and its derivatives), Cy3, CyS, Texas Red, Fluorescein, and the Alexa dyes (e.g. Alexa 568). Further fluorescent labels are available e.g. from Molecular Probes (Oregon). Also the use of quantum dots as fluorescent labels is contemplated. A radioactive label can be detected by any method known and appropriate, e.g. a light-sensitive film or a phosphor imager.

The amount of a polypeptide may be, also preferably, determined as follows: (a) contacting a solid support comprising a binding agent for the polypeptide as described elsewhere herein with a sample comprising the peptide or polypeptide and (b) measuring the amount of peptide or poly-peptide which is bound to the support. Materials for manufacturing supports are well-known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc.

In yet an aspect the sample is removed from the complex formed between the binding agent and the at least one marker prior to the measurement of the amount of formed complex. Accordingly, in an aspect, the binding agent may be immobilized on a solid support. In yet an aspect, the sample can be removed from the formed complex on the solid support by applying a washing solution.

“Sandwich assays” are among the most useful and commonly used assays encompassing a number of variations of the sandwich assay technique. Briefly, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Any unreacted material may be washed away, and the presence of the biomarker is determined by observation of a signal produced by the reporter molecule bound to the detection binding agent. The results may either be qualitative, by simple observation of a visible signal, or may be quantitated by comparison with a control sample containing known amounts of biomarker.

The incubation steps of a typical sandwich assays can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample which is capable of being specifically bound. Further details on how the measurement can be carried out are also found elsewhere herein. The amount of formed complex shall be transformed into an amount of the biomarker reflecting the amount indeed present in the sample.

The terms “binding agent”, “specific binding agent”, “analyte-specific binding agent”, “detection agent” and “agent that specifically binds to a biomarker” are used interchangeably herein. Preferably it relates to an agent that comprises a binding moiety which specifically binds the corresponding biomarker. Examples of “binding agents”, “detection agents”, “agents” are a nucleic acid probe, nucleic acid primer, DNA molecule, RNA molecule, aptamer, antibody, antibody fragment, peptide, peptide nucleic acid (PNA) or chemical compound. A preferred agent is an antibody which specifically binds to the biomarker to be determined. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity (i.e. antigen-binding fragments thereof). Preferably, the antibody is a polyclonal antibody (or an antigen-binding fragment therefrom). More preferably, the antibody is a monoclonal antibody (or an antigen binding fragment therefore Moreover, as described elsewhere herein, it is envisaged that two monoclonal antibodies are used that bind at different positions of TFPI-2 (in a sandwich immunoassay). Thus, at least one antibody is used for the determination of the amount of TFPI-2

In an embodiment, the at least one antibody is a mouse monoclonal antibody. In another embodiment, the at least one antibody is a rabbit monoclonal antibody. In a further embodiment, the antibody is goat polyclonal antibody. In an even further embodiment, the antibody is a sheep polyclonal antibody.

The term “specific binding” or “specifically bind” refers to a binding reaction wherein binding pair molecules exhibit a binding to each other under conditions where they do not significantly bind to other molecules. The term “specific binding” or “specifically binds”, when referring to a protein or peptide as biomarker, preferably refers to a binding reaction wherein a binding agent binds to the corresponding biomarker with an affinity (“association constant” Kₐ) of at least 10⁷ M⁻¹. The term “specific binding” or “specifically binds” preferably refers to an affinity of at least 10⁸ M⁻¹ or even more preferred of at least 10⁹ M⁻¹ for its target molecule. The term “specific” or “specifically” is used to indicate that other molecules present in the sample do not significantly bind to the binding agent specific for the target molecule.

In one embodiment, the method of the present invention is based on detecting a protein complex comprising human TFPI-2 and a non-human or chimeric TFPI-2-specific binding agent. In such embodiment the present invention reads on a method for assessing atrial fibrillation in a subject, said method comprising the steps of (a) incubating a sample from said subject with a non-human TFPI-2-specific binding agent (b) measuring the complex between the TFPI-2-specific binding agent and TFPI-2 formed in (a), and (c) comparing the measured amount complex to a reference amount. An amount of the complex at or above the reference amount is indicative for the diagnosis (and thus the presence) of atrial fibrillation, the presence of persistent atrial fibrillation, a subject who shall be subjected to ECG, or a subject who is at risk of an adverse event. An amount of the complex below the reference amount is indicative for the absence of atrial fibrillation, the presence of paroxysmal atrial fibrillation, a subject who is shall be not subjected to ECG, or a subject who is not at risk of an adverse event.

The term “amount” as used herein encompasses the absolute amount of a biomarker as referred to herein (such as TFPI-2 or the natriuretic peptide), the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts determined from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the biomarker (such as TFPI-2 and the natriuretic peptide such as NT-proBNP or BNP) in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a first sample. The comparison may be carried out manually or computer-assisted. Thus, the comparison may be carried out by a computing device. The value of the determined or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired assessment in a suitable output format.

In accordance with the present invention, the amount of the biomarker TFPI-2 and optionally the amount of the natriuretic peptide shall be compared to a reference. The reference is preferably a reference amount. The term “reference amount” is well understood by the skilled person. It is to be understood that the reference amount shall allow for the herein described assessment of atrial fibrillation. E.g., in connection with the method for diagnosing atrial fibrillation, the reference amount preferably refers to an amount which allows for allocation of a subject into either (i) the group of subjects suffering from atrial fibrillation or (ii) the group of subjects not suffering from atrial fibrillation. A suitable reference amount may be determined from a first sample to be analyzed together, i.e. simultaneously or subsequently, with the test sample.

It is to be understood that the amount of TFPI-2 is compared to a reference amount for TFPI2, whereas the amount of the natriuretic peptide is compared to a reference amount of the natriuretic peptide. If the amounts of two markers are determined, it is also envisaged that a combined score is calculated based on the amounts of TFPI-2 and the at least one further biomarker. In a subsequent step, the score is compared to a reference score.

Reference amounts can, in principle, be calculated for a cohort of subjects as specified above based on the average or mean values for a given biomarker by applying standard methods of statistics. In particular, accuracy of a test such as a method aiming to diagnose an event, or not, is best described by its receiver-operating characteristics (ROC) (see especially Zweig M H. et al., Clin. Chem. 1993; 39:561-577). The ROC graph is a plot of all the sensitivity versus specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of number of true-positive test results to the product of number of true-positive and number of false-negative test results. It is calculated solely from the affected subgroup. On the x-axis is the false-positive fraction, or 1-specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis for a given event with a proper balance of sensitivity and specificity, respectively. Accordingly, the reference to be used for the method of the present invention, i.e. a threshold which allows to assess atrial fibrillation can be generated, preferably, by establishing a ROC for said cohort as described above and deriving a threshold amount therefrom. Dependent on a desired sensitivity and specificity for a diagnostic method, the ROC plot allows deriving a suitable threshold. It will be understood that an optimal sensitivity is desired for e.g. excluding a subject from suffering from atrial fibrillation (i.e. a rule out) whereas an optimal specificity is envisaged for a subject to be assessed as suffering from atrial fibrillation (i.e. a rule in). In an embodiment, the method of the present invention allows for the prediction that a subject is at risk of an adverse event associated with atrial fibrillation such as the occurrence or recurrence of Atrial Fibrillation and/or stroke.

In a preferred embodiment, the term “reference amount” herein refers to a predetermined value. Said predetermined value shall allow for assessing atrial fibrillation, and thus for diagnosing atrial fibrillation, for differentiating between paroxysmal and persistent atrial fibrillation, for prediction the risk of an adverse event associated with atrial fibrillation, for identifying a subject who shall be subjected to electrocardiography (ECG), or for the assessment of a therapy for atrial fibrillation. It is to be understood that the reference amount may differ based on the type of assessment. E.g., the reference amount for TFPI-2 for the differentiation of AF will be usually higher than the reference amount for the diagnosis of AF. However, this will be taken into account by the skilled person.

As set forth above, the term “assessing atrial fibrillation” preferably refers to the diagnosis of atrial fibrillation, the differentiation between paroxysmal and persistent atrial fibrillation, the prediction of a risk of an adverse event associated with atrial fibrillation, to the identification of a subject who shall be subjected to electrocardiography (ECG), or the assessment of a therapy for atrial fibrillation. In the following, these embodiments of the method of the present invention will be described in more detail. The definitions above apply accordingly.

Method for Diagnosing Atrial Fibrillation

The term “diagnosing” as used herein means assessing whether a subject as referred to in accordance with the method of the present invention suffers from atrial fibrillation (AF), or not. In an embodiment, it is diagnosed that a subject suffers from AF. In a preferred embodiment, it is diagnosed that a subject suffers from paroxysmal AF. In an alternative embodiment, it is diagnosed that a subject does not suffer from AF.

In accordance with the present invention, all types of AF can be diagnosed. Thus, the atrial fibrillation may be paroxysmal, persistent or permanent AF. Preferably, persistent atrial fibrillation are diagnosed, in particular in a subject not suffering from permanent AF.

The actual diagnosis whether a subject suffers from AF, or not may comprise further steps such as the confirmation of a diagnosis (e.g. by ECG such as Holter-ECG). Thus, the present invention allows for assessing the likelihood that a patient suffers from atrial fibrillation. A subject who has an amount of TFPI-2 above the reference amount is likely to suffer from atrial fibrillation, whereas a subject who has an amount of TFPI-2 below the reference amount is not likely to suffer from atrial fibrillation. Accordingly, the term “diagnosing” in the context of the present invention also encompasses aiding the physician to assess whether a subject suffers from atrial fibrillation, or not.

Preferably, an amount of TFPI-2 (and optionally an amount of the at least one further biomarker such as ESM-1, Ang-2, IGFBP-7 and/or the natriuretic peptide) in the sample from a test subject which is (are) increased as compared to the reference amount (or to the reference amounts) is indicative for a subject suffering from atrial fibrillation, and/or an amount of TFPI-2 (and optionally an amount of the at least one further biomarker such as ESM-1, Ang-2, IGFBP-7 and/or the natriuretic peptide) in the sample from a subject which is (are) decreased as compared to the reference amount (or the reference amounts) is indicative for a subject not suffering from atrial fibrillation.

In a preferred embodiment, the reference amount, i.e. the reference amount TFPI-2 and, if is determined, the reference amount for the at least one further biomarker, shall allow for differentiating between a subject suffering from atrial fibrillation and a subject not suffering from atrial fibrillation. Preferably, said reference amount is a predetermined value.

In an embodiment, the method of the present invention allows for the diagnosis of a subject suffering from atrial fibrillation. Preferably, the subject is suffering from AF, if the amount of TFPI-2 (and optionally an amount of the at least one further biomarker such as ESM-1, Ang-2, IGFBP-7 and/or the natriuretic peptide) is (are) above the reference amount. In an embodiment, the subject is suffering from AF, if the amount of TFPI-2 is above a certain percentile (e.g. 99^{th} percentile) upper reference limit (URL) of a reference amount.

In another preferred embodiment, the method of the present invention allows for the diagnosis that a subject is not suffering from atrial fibrillation. Preferably, the subject is not suffering from AF, if the amount of TFPI-2 (and optionally an amount of the at least one further biomarker such as ESM-1, Ang-2, IGFBP-7 and/or the natriuretic peptide) is (are) below the reference amount (such as the certain percentile URL). Thus, in an embodiment, the term “diagnosing atrial fibrillation” refers to “ruling out atrial fibrillation”.

Ruling-out atrial fibrillation is of particular interest since further diagnostic tests for the diagnosis of atrial fibrillation such as an ECG test can be avoided. Thus, thanks to the present invention, unnecessary health care costs can be avoided.

Accordingly, the present invention also concerns a method for ruling out atrial fibrillation, comprising the steps of
a) determining the amount of TFPI-2 in a sample from the subject, and
b) comparing the amount of TFPI-2 to a reference amount whereby atrial fibrillation is ruled out.

Preferably, an amount of a the biomarker TFPI-2 in the sample of the subject which is decreased as compared to the reference amount (such as a reference for ruling out atrial fibrillation) is indicative for a subject who does not suffer from atrial fibrillation, and thus for ruling out atrial fibrillation in the subject. E.g. the reference amount for TFPI-2 may be determined in a sample from a subject who does not suffer from AF, or a from samples of a group thereof.

When determining the biomarker TFPI-2 and the natriuretic peptide in combination, an even more reliable rule-out can be achieved. Accordingly, steps a) and b) are preferably as follows:
a) determining the amount of TFPI-2 and the amount of a natriuretic peptide in a sample from the subject, and
b) comparing the amounts of TFPI-2 and the natriuretic peptide to reference amounts whereby atrial fibrillation is ruled out.

Preferably, amounts of both biomarkers, i.e. the amount of the biomarker TFPI-2 and the amount of the at least one further biomarker, in the sample of the subject which are decreased as compared to the respective reference amount (such as a reference amount for ruling out atrial fibrillation) are indicative for a subject who does not suffer from atrial fibrillation, and thus for ruling out atrial fibrillation in the subject. E.g. the reference amount for the natriuretic peptide may be determined in a sample from a subject who does not suffer from AF, or a from samples of a group thereof.

In an embodiment of the method of diagnosing atrial fibrillation, said method further comprises a step of recommending and/or initiating a therapy for atrial fibrillation based on the results of the diagnosis. Preferably, a therapy is recommended or initiated if it is diagnosed that the subject suffers from AF. Preferred therapies for atrial fibrillation are disclosed elsewhere herein.

Method for Differentiating Between Paroxysmal and Persistent Atrial Fibrillation

The term “differentiating” as used herein means to distinguish between paroxysmal and persistent atrial fibrillation in a subject. The term as used herein, preferably, includes differentially diagnosing paroxysmal and persistent atrial fibrillation in a subject. Thus, the method of the present invention allows for assessing whether a subject with atrial fibrillation suffers from paroxysmal atrial fibrillation or persistent atrial fibrillation. The actual differentiation may comprise further steps such as the confirmation of the differentiation. Thus, the term “differentiation” in the context of the present invention also encompasses aiding the physician to differentiate between paroxysmal and persistent AF.

Preferably, an amount of TFPI-2 (and optionally an amount of the at least one further biomarker such as ESM-1, Ang-2, IGFBP-7 and/or the natriuretic peptide) in the sample from a subject which is (are) increased as compared to the reference amount (or to the reference amounts) is indicative for a subject suffering from persistent atrial fibrillation and/or an amount of TFPI-2 (and optionally an amount of the at least one further biomarker such as ESM-1, Ang-2, IGFBP-7 and/or the natriuretic peptide) in the sample from a subject which is (are) decreased as compared to a reference amount (or to the reference amounts) is indicative for a subject suffering from paroxysmal atrial fibrillation. In both AF types (paroxysmal and persistent), the amount of TFPI-2 is increased as compared to the reference amount of non-AF subjects.

In a preferred embodiment, the reference amount(s) shall allow for differentiating between a subject suffering from paroxysmal atrial fibrillation and a subject suffering from persistent atrial fibrillation. Preferably, said reference amount is a predetermined value.

In an embodiment of the above method of differentiating between paroxysmal and persistent atrial fibrillation, the subject does not suffer from permanent atrial fibrillation.

Method for Predicting the Risk a Risk of an Adverse Event Associated with Atrial Fibrillation

The method of the present invention also contemplates a method for predicting the risk of an adverse event.

In an embodiment, the risk of an adverse event as set forth herein can be the prediction of any adverse event associated with atrial fibrillation. Preferably, said adverse event is selected from recurrence of atrial fibrillation (such as the recurrence of atrial fibrillation after cardioversion) and stroke. Accordingly, the risk of a subject (who suffers from atrial fibrillation) to suffer in the future from an adverse event (such as stroke or recurrence of atrial fibrillation) shall be predicted.

Further, it is envisaged that said adverse event associated with atrial fibrillation is the occurrence of atrial fibrillation in a subject has no known history of atrial fibrillation.

In a particularly preferred embodiment, the risk of stroke is predicted.

Accordingly, the present invention method for predicting the risk of stroke in a subject, comprising the steps of
a) determining the amount of TFPI-2 in a sample from the subject, and
b) comparing the amount of TFPI-2 to a reference amount, whereby the risk of stroke is to be predicted.

In particular, the present invention relates to a method for predicting the risk of stroke in a subject, comprising the steps of
(a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
(b) comparing the amount of the biomarker TFPI-2 to a reference amount for TFPI-2 and, optionally, comparing the amount of the at least one further biomarker to a reference amount for said at least one further biomarker, whereby the risk of stroke is to be predicted.

Preferably, term “predicting the risk” as used herein refers to assessing the probability according to which the subject will suffer from an adverse event as referred to herein (e.g. of stroke). Typically, it is predicted whether a subject is at risk (and thus at elevated risk) or not at risk (and thus at reduced risk) of suffering from said adverse event. Accordingly, the method of the present invention allows for differentiating between a subject at risk and a subject not at risk of suffering from said adverse event. Further, it is envisaged that the method of the present invention allows for differentiating between a subject who is a reduced, average, or elevated risk.

As set forth above, the risk (and probability) of suffering from said adverse event within a certain time window shall be predicted. In a preferred embodiment of the present invention, the predictive window is a period of about three months, about six months, or, in particular, about one year. Thus, the short-term risk is predicted.

In another preferred embodiment, the predictive window is a period of about five years (e.g. for the prediction of stroke). Further, the predictive window might be a period of about six years (e.g. for the prediction of stroke). Alternatively, the predictive window may be about 10 years. Also, it is envisaged that the predictive window a period of 1 to 3 years. Thus, the risk to suffer from stroke within 1 to 3 year is predicted. Also, it is envisaged that the predictive window a period of 1 to 10 years. Thus, the risk to suffer from stroke within 1 to 10 years is predicted.

Preferably, said predictive window is calculated from the completion of the method of the present invention. More preferably, said predictive window is calculated from the time point at which the sample to be tested has been obtained. As will be understood by those skilled in the art, the prediction of a risk is usually not intended to be correct for 100% of the subjects. The term, however, requires that prediction can be made for a statistically significant portion of subjects in a proper and correct manner Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.1, 0.05, 0.01, 0.005, or 0.0001.

In a preferred embodiment, the expression “predicting the risk of suffering from said adverse event” means that the subject to be analyzed by the method of the present invention is allocated either into the group of subjects being at risk of suffering from said adverse event, or into the group of subjects not being at risk of suffering from said adverse event (such as stroke). Thus, it is predicted whether the subject is at risk or not at risk of suffering from said adverse event. As used herein “a subject who is at risk of suffering from said adverse event”, preferably has an elevated risk of suffering from said adverse event (preferably within the predictive window). Preferably, said risk is elevated as compared to the average risk in a cohort of subjects. As used herein, “a subject who is not at risk of suffering from said adverse event”, preferably, has a reduced risk of suffering from said adverse event (preferably within the predictive window). Preferably, said risk is reduced as compared to the average risk in a cohort of subjects. A subject who is at risk of suffering from said adverse event preferably has a risk of suffering from said adverse event such as recurrence or occurrence of atrial fibrillation of at least 20% or more preferably of at least 30%, preferably, within a predictive window of about one year. A subject who is not at risk of suffering from said adverse event preferably has a risk of lower than 12%, more preferably of lower than 10% of suffering from said adverse event, preferably within a predictive window of one year.

With respect to the prediction of stroke, a subject who is at risk of suffering from said adverse event preferably has a risk of suffering from said adverse event of at least 10% or more preferably of at least 13%, preferably, within a predictive window of about five years, or in particular of about six years. A subject who is not at risk of suffering from said adverse event preferably has a risk of lower than 10%, more preferably of lower than 8%, or most preferably of lower than 5% of suffering from said adverse event, preferably within a predictive window of about five years, or in particular of about six years. The risk may be higher, if the subject does not receive anticoagulation therapy. This will be taken into account by the skilled person.

Preferably, an amount of TFPI-2 (and optionally an amount of the at least one further biomarker such as ESM-1, Ang-2, IGFBP-7 and/or the natriuretic peptide) in the sample from a subject which is (are) increased as compared to the reference amount (or to the reference amounts) is indicative for a subject who is at risk of the adverse event associated with atrial fibrillation. and/or an amount of TFPI-2 (and optionally an amount of the at least one further biomarker such as ESM-1, Ang-2, IGFBP-7 and/or the natriuretic peptide) in the sample from a subject which is decreased as compared to the reference amount (or to the reference amounts) is indicative for a subject who is not at risk the adverse event associated with atrial fibrillation.

In a preferred embodiment, the reference amount (or reference amounts) shall allow for differentiating between a subject who is at risk of an adverse event as referred to herein and a subject who is not at risk of said adverse event. Preferably, said reference amount is a predetermined value.

In accordance with the method of the present invention, the risk of stroke shall be predicted. The term “stroke” is well known in the art. As used herein, the term, preferably, refers to ischemic stroke, in particular to cerebral ischemic stroke. A stroke which is predicted by the method of the present invention shall be caused by reduced blood flow to the brain or parts thereof which leads to an undersupply of oxygen to brain cells. In particular, the stroke leads to irreversible tissue damage due to brain cell death. Symptoms of stroke are well known in the art. Ischemic stroke may be caused by atherothrombosis or embolism of a major cerebral artery, by coagulation disorders or nonatheromatous vascular disease, or by cardiac ischemia which leads to a reduced overall blood flow. The ischemic stroke is preferably selected from the group consisting of atherothrombotic stroke, cardioembolic stroke and lacunar stroke. Preferably, the stroke to be predicted is an acute ischemic stroke, in particular cardioembolic stroke. A cardioembolic stroke (frequently also referred to as embolic or thromboembolic stroke) can be caused by atrial fibrillation.

Preferably, said stroke shall be associated with atrial fibrillation. More preferably, the stroke shall be caused by atrial fibrillation. However, it is also envisaged that the subject has no history of atrial fibrillation.

Preferably, a stroke is associated with atrial fibrillation, if there is a temporal relationship between the stroke and an episode of atrial fibrillation. More preferably, a stroke is associated with atrial fibrillation, if the stroke is caused by atrial fibrillation. Most preferably, a stroke is associated with atrial fibrillation, if the stroke can be caused by atrial fibrillation. For example, a cardioembolic stroke (frequently also referred to as embolic or thromboembolic stroke) can be caused by atrial fibrillation. Preferably, a stroke associated with AF can be prevented by oral anticoagulation.

Also preferably, the stroke is considered as associated with atrial fibrillation, if the subject to be tested suffers from atrial fibrillation and/or has a known history thereof. Also, in an embodiment, the stroke may be considered as being associated with atrial fibrillation, if the subject is suspected to suffer from atrial fibrillation.

The term “stroke” does, preferably, not include hemorrhagic stroke. Whether a subject suffers from stroke, in particular from ischemic stroke can be determined by well known methods. Moreover, symptoms of stroke are well known in the art. E.g., stroke symptoms include sudden numbness or weakness of face, arm or leg, especially on one side of the body, sudden confusion, trouble speaking or understanding, sudden trouble seeing in one or both eyes, and sudden trouble walking, dizziness, loss of balance or coordination.

The terms “comparing”, “amount”, “subject”, and “determining” etc. are defined elsewhere herein. The definitions apply accordingly. E.g. the sample is preferably a blood, serum or plasma sample.

In a preferred embodiment of the aforementioned method of predicting an adverse event (such as stroke), the subject to be tested suffers from atrial fibrillation. More preferably, the subject has a known history of atrial fibrillation. In accordance with the method for predicting an adverse event, the subject preferably suffers from permanent atrial fibrillation, more preferably from persistent atrial fibrillation and most preferably from paroxysmal atrial fibrillation.

In an embodiment of the method of predicting an adverse event, the subject suffering from atrial fibrillation experiences episodes of atrial fibrillation when the sample is obtained. In another embodiment of the method of predicting an adverse event, the subject suffering from atrial fibrillation does not experiences episode of atrial fibrillation when the sample is obtained (and thus shall have a normal sinus rhythm). Further, the subject whose risk is to be predicted may be on anticoagulation therapy.

In another preferred embodiment of the aforementioned method of predicting an adverse event (such as stroke), the subject to be tested has a history of stroke.

In another preferred embodiment of the aforementioned method of predicting an adverse event (such as stroke), the subject to be tested has a history of thromboembolism.

In another embodiment of the method of predicting an adverse event, the subject to be tested has no known history of atrial fibrillation. In particular, it is envisaged that the subject does not suffer from atrial fibrillation.

The method of the present invention may aid personalized medicine. In a preferred embodiment, the method for predicting the risk of stroke in a subject further comprises i) the step of recommending anticoagulation therapy, or ii) of recommending an intensification of anticoagulation therapy, if the subject has been identified to be at risk to suffer from stroke.

In another preferred embodiment, the method for predicting the risk of stroke in a subject further comprises i) the step of initiating anticoagulation therapy, or ii) of intensifying anticoagulation therapy, if the subject has been identified to be at risk to suffer from stroke (by the method of the present invention).

If the test subject is on anticoagulation therapy, and if the subject has been identified not to be at risk to suffer from stroke (by the method of the present invention) the dosage of anticoagulation therapy may be reduced. Accordingly, a reduction of the dosage may be recommended. Be reducing the dosage, the risk to suffer from side effects (such as bleeding) may be reduced.

The term “recommending” as used herein means establishing a proposal for a therapy which could be applied to the subject. The therapy to be recommended depends on the outcome of the provided by the method of the present invention.

In particular, the following applies:

If the subject to be tested does not receive anticoagulation therapy, the initiation of anticoagulation is recommended, if the subject has been identified to be at risk to suffer from stroke. Thus, anticoagulation therapy shall be initiated.

If the subject to be tested already receives anticoagulation therapy, the intensification of anticoagulation is recommended, if the subject has been identified to be at risk to suffer from stroke. Thus, anticoagulation therapy shall be intensified.

In a preferred embodiment, anticoagulation therapy is intensified by increasing the dosage of the anticoagulant, i.e. the dosage of the currently administered coagulant.

In a particularly preferred embodiment, anticoagulation therapy is intensified by increasing the replacing the currently administered anticoagulant with a more effective anticoagulant. Thus, a replacement of the anticoagulant is recommended.

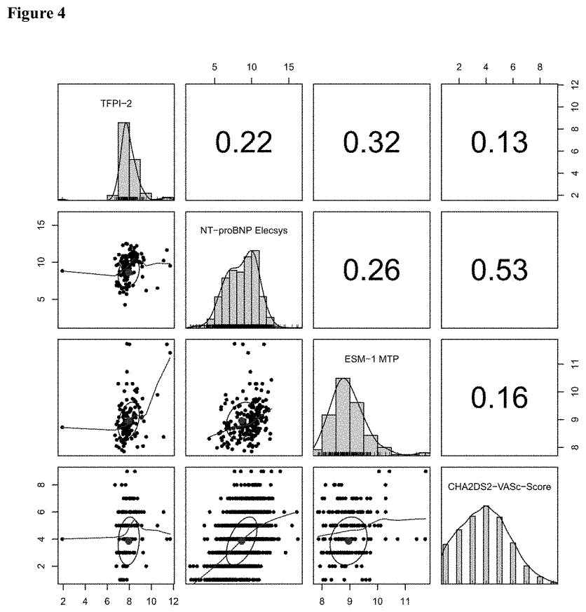

It has been described that better prevention in high risk patients is achieved with the oral anticoagulant apixaban versus the vitamin K antagonist warfarin as shown in Hijazi at al., The Lancet 2016 387, 2302-2311, (FIG. 4).

Thus, it is envisaged that the subject to be tested is a subject who is treated with a vitamin K antagonist such as warfarin or dicumarol. If the subject has been identified to be at risk to suffer from stroke (by the method of the present invention), the replacement of the vitamin K antagonist with an oral anticoagulant, in particular dabigatran, rivaroxaban or apixaban is recommended. Accordingly, the therapy with the vitamin K antagonist is discontinued and therapy with an oral anticoagulant is initiated.

In the studies underlying the present invention, it has been further shown that the determination of TFPI-2 allows for improving the prediction accuracy of a clinical stroke risk score for a subject. Thus, the combined determination of clinical stroke risk score and the determination of TFPI-2 allows for an even more reliable prediction of stroke as compared to the determination of TFPI-2 or the determination of the clinical stroke risk score alone. (see Example 3).

Accordingly, the method for predicting the risk of stroke may further comprise the combination of the amount of TFPI-2 with the clinical stroke risk score. Based on the combination of the amount of TFPI-2 and the clinical risk score, the risk of stroke of the test subject is predicted.

In an embodiment of the aforementioned method, the method further comprises the comparison of the amount of TFPI-2 with a reference amount. In this case, the results of the comparison is combined with the clinical stroke risk score.

Accordingly, the present invention, in particular, relates to method for predicting the risk of stroke in a subject, comprising the steps of
a) determining the amount of TFPI-2 in a sample from the subject, and
b) combining the amount of TFPI-2 with a clinical stroke risk score, whereby the risk of stroke of said subject is to be predicted.

In accordance with this method, it is envisaged that the subject is a subject who has a known clinical stroke risk score. Accordingly, the value for the clinical stroke risk score is known for the subject.

Alternatively, the method may comprise obtaining or providing the value for the clinical stroke risk score. Preferably, the value is a number. In an embodiment, the clinical stroke risk score is generated by one of the clinically based tools available to physicians. Preferably, the value provided by determining the value for the clinical stroke risk score for the subject. More preferably, the value is obtained from patient record databases and medical history of the subject. The value for the score therefore can be also determined using historical or published data of the subject.

In accordance with the present invention, the amount of TFPI-2 is combined with the clinical stroke risk score. This means preferably, that the value for the amount of TFPI-2 is combined with the clinical stroke risk score. Accordingly, the values are operatively combined to predict the risk of the subject to suffer from stroke. By combining the value, a single value may be calculated, which itself can be used for the prediction.

Clinical stroke risk scores are well known in the art. E.g. said scores are described in Kirchhof P. et al., (European Heart Journal 2016; 37: 2893-2962) which herewith is incorporated by references with respect to its entire disclosure content. In an embodiment, the score is CHA₂DS₂-VASc-Score. In another embodiment, the score is the CHADS₂ Score. (Gage B F. Et al., JAMA, 285 (22) (2001), pp. 2864-2870) and ABC score (Hijazi Z. et al., Lancet 2016; 387(10035): 2302-2311)

Method for Improving the Prediction Accuracy of a Clinical Stroke Risk Score

The present invention further relates to a method for improving the prediction accuracy of a clinical stroke risk score for a subject, comprising the steps of
a) determining the amount of TFPI-2 in a sample, and
b) combining a value for the amount of TFPI-2 with the clinical stroke risk score, whereby the prediction accuracy of said clinical stroke risk score is improved.

The method may comprise the further step of c) improving prediction accuracy of said clinical stroke risk score based on the results of step b).

In a preferred embodiment, steps a) and b) of the aforementioned method are as follows:
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), wherein the subject has a known clinical stroke risk score, and
b) combining a value for the amount of TFPI-2 and/or the amount of one or more biomarkers comprising of a natriuretic peptide, ESM-1, Ang2, IGFBP7 with the clinical stroke risk score, whereby the prediction accuracy of said clinical stroke risk score is improved.

The definitions and explanations given herein above in connection with the method of assessing atrial fibrillation, in particular of predicting the risk of an adverse event (such as stroke) preferably apply to the aforementioned method as well E.g., it envisaged that the subject is a subject who has a known clinical stroke risk score. Alternatively, the method may comprise obtaining or providing the value for the clinical stroke risk score.

In accordance with the present invention, the amount of TFPI-2 is combined with the clinical stroke risk score. This means preferably, that the value for the amount of TFPI-2 is combined with the clinical stroke risk score. Accordingly, the values are operatively combined to improve the prediction accuracy of said clinical stroke risk score.

Method for Identifying a Subject Who Shall be Subjected to Electrocardiography (ECG)

In accordance with this embodiment of method of the present invention, it shall be assessed whether the subject to be tested with the biomarker shall be subjected to electrocardiography (ECG), i.e. to an electrocardiography assessment. Said assessment shall be carried for diagnosing, i.e. to detect the presence of absence of AF, in said subject.

The term “identifying a subject” as used herein preferably refers to using the information or data generated relating to the amount of TFPI-2 in a sample of a subject to identify subject shall be subjected to ECG. The subject who is identified has an increased likelihood of suffering from AF. The ECG assessment is made as a confirmation.

Electrocardiography (abbreviated ECG) is the process of recording the electrical activity of the heart by suitable ECG. An ECG device records the electrical signals produced by the heart which spread throughout the body to the skin. The recording is of the electrical signal is achieved by contacting the skin of the test subject with electrodes comprised by the ECG device. The process of obtaining the recording is non-invasive and risk-free. The ECG is carried out for the diagnosis of atrial fibrillation, i.e. for the assessment of the presence of absence of atrial fibrillation in the test subject. In embodiment of the method of the present invention, the ECG device is a one-lead device (such as a one-lead handheld ECG-device). In another preferred embodiment the ECG device is a 12-lead ECG device such as a Holter monitor.

Preferably, an amount of TFPI-2 (and optionally an amount of the at least one further biomarker such as ESM-1, Ang-2, IGFBP-7 and/or the natriuretic peptide) in the sample from a test subject which is (are) increased as compared to the reference amount (or to the reference amounts) is indicative for a subject who shall be subjected to ECG, and/or an amount of TFPI-2 (and optionally an amount of the at least one further biomarker such as ESM-1, Ang-2, IGFBP-7 and/or the natriuretic peptide) in the sample from a subject which is (are) decreased as compared to the reference amount (or to the reference amounts) is indicative for a subject who shall not be subjected to ECG.

In a preferred embodiment, the reference amount shall allow for differentiating between a subject who shall be subjected to ECG and a subject who shall not be subjected to ECG. Preferably, said reference amount is a predetermined value.

Method for the Assessment of a Therapy for Atrial Fibrillation

As used herein, the term “assessing a therapy for atrial fibrillation”, preferably refers to the assessment of a therapy that aims to treat atrial fibrillation. In a preferred embodiment, said therapy is anticoagulation therapy. Accordingly, the present invention encompasses a method for assessing anticoagulation therapy.

For example, the efficacy of a therapy (e.g. anticoagulation therapy) shall be assessed. The assessment of a therapy for atrial fibrillation includes the monitoring of a therapy (e.g. anticoagulation therapy). Further, the assessment of a therapy for atrial fibrillation includes the identification of a subject being eligible to the administration of at least one anticoagulation medicament or being eligible to an intensified anticoagulation therapy. The subject in accordance with the assessment of a therapy for atrial fibrillation may or may not suffer from atrial fibrillation.

The therapy to be assessed can be any therapy that aims to treat atrial fibrillation. Preferably, said therapy is selected from the group consisting of administration of at least one anticoagulant (i.e. anticoagulation therapy), rhythm control, rate control, cardioversion and ablation. Said therapies are well known in the art and are e.g. reviewed in Fuster V et al. Circulation 2011; 123:e269-e367 which herewith is incorporated by reference in its entirety.

In an embodiment, the therapy is the administration of at least one anticoagulant, i.e. anticoagulation therapy. Anticoagulation therapy is preferably a therapy which aims to reduce the risk of anticoagulation in said subject. Administration of at least one anticoagulant shall aim to reduce or prevent coagulation of blood and related stroke. In a preferred embodiment, at least one anticoagulant is selected from the group consisting of heparin, a coumarin derivative (i.e. a vitamin K antagonist), in particular warfarin or dicumarol, oral anticoagulants, in particular dabigatran, rivaroxaban or apixaban, tissue factor pathway inhibitor (TFPI), antithrombin III, factor IXa inhibitors, factor Xa inhibitors, inhibitors of factors Va and VIIIa and thrombin inhibitors (anti-IIa type). Accordingly, it is envisaged that the subject takes at least one of the aforementioned medicaments.

In preferred embodiment, the anticoagulant is a vitamin K antagonist such as warfarin or dicumarol. Vitamin K antagonists, such as warfarin or dicumarol are less expensive, but need better patient compliance, because of the inconvenient, cumbersome and often unreliable treatment with fluctuating time in therapeutic range. NOAC (new oral anticoagulants) comprise direct factor Xa inhibitors (apixaban, rivaroxaban, darexaban, edoxaban), direct thrombin inhibitors (dabigatran) and PAR-1 antagonists (vorapaxar, atopaxar).

In another preferred embodiment the anticoagulant is an oral anticoagulant, in particular apixaban, rivaroxaban, darexaban, edoxaban, dabigatran, vorapaxar, or atopaxar.

Thus, the subject to be tested may be on therapy with an oral anticoagulant or a vitamin K antagonist at the time of the testing (i.e. at the time at which the sample is received.

In a preferred embodiment, the assessment of a therapy for atrial fibrillation is the monitoring of said therapy. In this embodiment, the reference amount is preferably the amount for TFPI2 in an earlier obtained sample (i.e. in a sample that has been obtained prior to the test sample in step a), herein also referred to as the first sample.

Optionally, the amount of the at least one further biomarker as referred to herein is determined in addition to the amount of TFPI-2.

Accordingly, the present invention relates to a method for monitoring a therapy for atrial fibrillation, such as a method of monitoring anticoagulation therapy, in a subject, wherein said method comprises the steps of
(a) determining, in at first sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7),
(b) determining, in a second sample from the subject, the amount of the biomarker TFPI2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), wherein said second sample has been obtained after said first sample,
(c) comparing the amount of TFPI-2 in the first sample to the amount of TFPI-2 in said second sample, and optionally comparing the amount of said at least one further biomarker in the first sample to the amount of said at least one further biomarker in said second sample, thereby monitoring the therapy, such as anticoagulation therapy.

In an embodiment of the above method, the subject suffers from atrial fibrillation. In another embodiment of the above method, the subject does not suffer from atrial fibrillation.

The term “monitoring” as used herein, preferably, relates to assessing the effects a therapy as referred to herein elsewhere. Thus, the efficacy of a therapy (such as anticoagulation therapy) is monitored.

The aforementioned method may comprise the further step of monitoring the therapy based on the results of the comparison step carried out in step c). As will be understood by those skilled in the art, the prediction of a risk is usually not intended to be correct for 100% of the subjects. The term, however, requires that prediction can be made for a statistically significant portion of subjects in a proper and correct manner Thus, the actual monitoring may comprise further steps such as the confirmation.

Preferably, by carrying out the method of the present invention it can be assessed whether the subject responds to said therapy or not. A subject responds to a therapy if the condition the subject improves between obtaining the first and the second sample. Preferably, a subject does not respond to the therapy if the condition worsened between obtaining the first and the second sample.

A subject is, preferably, considered to respond to the therapy, if the therapy reduces the risk of the subject of recurrence of atrial fibrillation. A subject is, preferably, considered as not to respond to the therapy, if the therapy does not reduce the risk of the subject of recurrence of atrial fibrillation.

Alternatively, a subject who responds to anticoagulation therapy, is preferably a subject whose risk of stroke decreases between obtaining the first and the second sample. A subject who does not respond to anticoagulation therapy, is preferably a subject whose risk of stroke increases, or remains unchanged between obtaining the first and the second sample. Whether the risk of stroke increases, decreases, or remains unchanged can be, e.g., determined by assessing the subject's clinical stroke risk score. Preferred scores are disclosed elsewhere herein.

Preferably, the first sample is obtained prior to the initiation of said therapy. More preferably, the sample is obtained within one week in particular within two weeks prior to the initiation of said therapy. However, it is also contemplated that the first sample may is obtained after initiation of said therapy (but before the second sample is obtained). In this case an ongoing therapy is monitored.

Thus, the second sample shall be obtained after the first sample. It is to be understood that the second sample shall be obtained after the initiation of said therapy.

Moreover, it is particularly contemplated that the second sample is obtained after a reasonable period of time after obtaining the first sample. It is to be understood, that the amounts of biomarkers referred herein, do not instantly change (e.g. within 1 minute or 1 hour) Therefore, “reasonable” in this context refers to intervals between obtaining the first and second sample which intervals allow the biomarker(s) to adjust. Therefore, the second sample, preferably, is obtained at least one month after said first sample, at least three months, or, in particular, at least six month after said first sample.

Preferably, a decrease and, more preferably, a significant decrease, and, most preferably, a statistically significant decrease of the amount(s) of the biomarker(s), i.e. of TFPI-2 and optionally of the natriuretic peptide in the second sample as compared to the amount(s) of the biomarker(s) in the first sample is indicative for a subject who responds to the therapy. Thus, the therapy is efficient. Also preferably, no change of the concentration of TFPI-2 or an increase, more preferably, a significant increase, most preferably, a statistically significant increase of the amount(s) of the biomarker(s) in the second sample as compared to the amount(s) of the biomarker(s) in the first sample is indicative for a subject who does not respond to the therapy. Thus, the therapy is not efficient.

The terms “significant” and “statistically significant” are known by the person skilled in the art. Thus, whether an increase or decrease is significant or statistically significant can be determined without further ado by the person skilled in the art using various well known statistic evaluation tools. For example, a significant increase or decrease is an increase or decrease of at least 10%, in particular of at least 20%.

In an embodiment, the intensity of the therapy is increased if the subject does not respond to the therapy. Moreover, it is envisaged that the intensity of the therapy is decreased, if a subject responds to the therapy. Alternatively, the therapy can be continued with the same intensity, if a subject responds to the therapy.

For example, the intensity of a therapy can be increased by increasing the dosage of the administered medicament. For example, the intensity of a therapy can be decreased by decreasing the dosage of the administered medicament. If a therapy is continued with the same intensity, the administered medicament and the dosage may remain unchanged. With respect to increasing the intensity of anticoagulation therapy, please see explanations made herein elsewhere, such as the explanations made in connection with the assessment of the efficacy of an anticoagulation therapy of a subject.

In another preferred embodiment, the assessment of a therapy for atrial fibrillation is the guidance of a therapy for atrial fibrillation. The term “guidance” as used herein, preferably, relates to adjusting the intensity of a therapy, such as increasing or decreasing the dose of oral anticoagulation, based on the determination of the biomarker, i.e. TFPI-2, during therapy.

In a further preferred embodiment, the assessment of a therapy for atrial fibrillation is the stratification of a therapy for atrial fibrillation. Thus, a subject shall be identified who is eligible to a certain therapy for atrial fibrillation. The term “stratification” as used herein, preferably, relates to selecting an adequate therapy based on the particular risk, molecular path identified and/or expected efficacy of the particular drug or procedure. Depending on the risk detected, particularly patients with minimal or no symptoms related to the arrhythmia will become eligible to control of the ventricular rate, cardioversion or ablation, who otherwise would receive only antithrombotic therapy.

The method of the present invention can be used for assessing the efficacy of an anticoagulation therapy of a subject. In an embodiment, the efficacy of an anticoagulation therapy is assessed by carrying out the following steps:

a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and

b) comparing the amount of the biomarker TFPI-2 to a reference amount for TFPI-2 and, optionally, comparing the amount of the at least one further biomarker to a reference amount for said at least one further biomarker, whereby the efficacy of the anticoagulation therapy is to be assessed.

Preferably, an amount of TFPI-2 (and optionally an amount of the at least one further biomarker such as ESM-1, Ang-2, IGFBP-7 and/or the natriuretic peptide) in the sample from a test subject which is (are) increased as compared to the reference amount (or to the reference amounts) indicates that the therapy is not effective, and/or an amount of TFPI-2 (and optionally an amount of the at least one further biomarker such as ESM-1, Ang-2, IGFBP-7 and/or the natriuretic peptide) in the sample from a subject which is (are) decreased as compared to the reference amount (or the reference amounts) indicates that the therapy is effective.

If the test subject receives an anticoagulation therapy and if the above method indicates that the therapy is not effective, the intensification of anticoagulation therapy is recommended. Thus, anticoagulation therapy shall be intensified or a replacement of the anticoagulant is recommended.

The method of the present invention can be used for identifying a subject being eligible to the administration of at least one anticoagulation medicament or being eligible to an intensified anticoagulation therapy. This can be achieved by carrying out the following steps:

a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and

b) comparing the amount of the biomarker TFPI-2 to a reference amount for TFPI-2 and, optionally, comparing the amount of the at least one further biomarker to a reference amount for said at least one further biomarker, whereby a subject being eligible to the administration of said at least one medicament or to an intensified anticoagulation therapy is identified.

Preferably, an amount of TFPI-2 (and optionally an amount of the at least one further biomarker such as ESM-1, Ang-2, IGFBP-7 and/or the natriuretic peptide) in the sample from a test subject which is (are) increased as compared to the reference amount (or to the reference amounts) indicates that the subject is eligible to the administration of at least one anticoagulation medicament or is eligible to an intensified anticoagulation therapy. An amount of TFPI-2 (and optionally an amount of the at least one further biomarker such as ESM-1, Ang-2, IGFBP-7 and/or the natriuretic peptide) in the sample from a subject which is (are) decreased as compared to the reference amount (or the reference amounts) that the subject is not eligible to the administration of at least one anticoagulation medicament or is not eligible to an intensified anticoagulation therapy. If the test subject is on anticoagulation therapy, and if the subject has been identified not to be eligible to an increase of at least one anticoagulation medicament the dosage of anticoagulation therapy may be even reduced.

The reference amount shall allow for differentiating between a subject who is eligible to the administration of at least one anticoagulation medicament and between a subject who is not eligible to the administration of at least one anticoagulation medicament. Alternatively or additionally, the reference amount shall allow for differentiating between a subject who is eligible to the intensification of anticoagulation therapy and between a subject who is not eligible to the intensification of anticoagulation therapy.

In particular, the following applies:

If the subject to be tested does not receive anticoagulation therapy, the aforementioned method is carried out for identifying a subject who is eligible to the administration of at least one anticoagulation medicament. The administration of at least one anticoagulation medicament is recommended, if the subject has been identified to eligible to the administration of at least one anticoagulation medicament. Thus, therapy with administration of at least one anticoagulation medicament shall be initiated.

If the subject to be tested already receives anticoagulation therapy (i.e. drug therapy e.g. the administration of a vitamin K antagonist such as warfarin or dicumarol), the aforementioned method is carried out for identifying a subject who is eligible to the intensification of anticoagulation therapy. The intensification of anticoagulation is recommended, if the subject has been identified to be eligible to the intensification of anticoagulation therapy.

In an embodiment, anticoagulation therapy is intensified by increasing the dosage of the anticoagulant(s), i.e. the dosage of the currently administered anticoagulant(s).

In another embodiment, anticoagulation therapy is intensified by replacing the currently administered anticoagulant(s) with a more effective anticoagulant. Thus, a replacement of the anticoagulant is recommended.

As set forth above, it has been described that better prevention in high risk patients is achieved with the oral anticoagulant apixaban versus the vitamin K antagonist warfarin as shown in Hijazi at al., The Lancet 2016 387, 2302-2311, (FIG. 4).

Accordingly, the subject to be tested may a subject who is treated with a vitamin K antagonist such as warfarin or dicumarol. If the subject has been identified to be eligible to the intensification of anticoagulation therapy, the subject is eligible to the replacement of the vitamin K antagonist with an oral anticoagulant, in particular dabigatran, rivaroxaban or apixaban. Accordingly, the therapy with the vitamin K antagonist is discontinued and therapy with an oral anticoagulant is initiated.

The present invention further concerns a method of aiding in the assessment of atrial fibrillation, said method comprising the steps of:
a) providing at least one sample from a subject,
b) determining, in the at least one sample provided in step a), the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 and IGFBP7 (Insulin-like growth factor-binding protein 7), and
c) providing information on the determined amount of the biomarker TFPI-2 and optionally on the determined amount of the at least one further biomarker to a physician, thereby aiding in the assessment of atrial fibrillation.

The physician shall be the attending physician, i.e. the physician who requested the determination of the biomarker(s). The aforementioned method shall aid the attending physician in the assessment of atrial fibrillation. Thus, the method does not encompass the diagnosis, prediction, monitoring, differentiation, identification as referred to above in connection with the method of assessing atrial fibrillation.

Step a) of the aforementioned method of obtaining the sample does not encompass the drawing of the sample from the subject. Preferably, the sample is obtained by receiving a sample from said subject. Thus, the sample can have been delivered.

In an embodiment, the method above is a method of aiding in the prediction of stroke, said method comprising the steps of:
a) providing at least one sample from a subject as referred to herein in connection with the method of assessing atrial fibrillation, in particular in connection with the method of predicting atrial fibrillation,
b) determining the amount of the biomarker TFPI-2 and the amount of at least one further biomarker selected from the group consisting of a natriuretic peptide, ESM-1 (Endocan), Ang2 and IGFBP7 (Insulin-like growth factor-binding protein 7), and
c) c) providing information on the determined amount of the biomarker TFPI-2 and optionally on the determined amount of the at least one further biomarker to a physician, thereby aiding in the prediction of stroke.

The present invention further relates to a method, comprising:
a) providing an assay for the biomarker TFPI-2 and, optionally, at least one further assay for a further biomarker selected from the group consisting of a natriuretic peptide, ESM-1 (Endocan), Ang2 and IGFBP7 (Insulin-like growth factor-binding protein 7), and
b) providing instructions for use of assay results obtained or obtainable by said assay(s) in the assessment of atrial fibrillation.

The purpose of the aforementioned method is, preferably, the aid in the assessment of atrial fibrillation.

The instructions shall contain a protocol for carrying out the method of assessing atrial fibrillation as described herein above. Further, the instructions shall contain at least one value for a reference amount for TFPI-2 and optionally at least one value for a reference amount for a natriuretic peptide.

The “assay” is preferably a kit adapted for determining the amount of the biomarker. The term “kit” is explained herein below. E.g. said kit shall comprise at least one detection agent for the biomarker TFPI-2 and optionally and at least one further agent selected from the group consisting of an agent which specifically binds to a natriuretic peptide, an agent which specifically binds to ESM-1, an agent which specifically binds Ang2 and an agent which specifically binds to IGFBP7. Thus, one to four detection agents may be present. The detection agents for the one to four biomarkers can be provided in a single kit or in separate kits.

The test result obtained or obtainable by said test, is the value for the amount of the biomarker(s).

In an embodiment, step b) comprises providing instructions for using of test results obtained or obtainable by said test(s) in prediction of stroke (as described herein elsewhere).

The present invention further pertains to computer-implemented method for assessing atrial fibrillation, comprising
d) receiving, at a processing unit, a value for the amount of TFPI-2, and, optionally at least one further value for the amount of at least one further biomarker selected from the group consisting of a natriuretic peptide, ESM-1 (Endocan), Ang2 and IGFBP7 (Insulin-like growth factor-binding protein 7), wherein said amount of TFPI-2 and, optionally, the amount of the at least one further biomarker have been determined in a sample from a subject,
e) comparing, by said processing unit, the value or values received in step (a) to a reference or to references, and
f) assessing atrial fibrillation based in the comparison step b).

The above-mentioned method is a computer-implemented method. Preferably, all steps of the computer-implemented method are performed by one or more processing units of a computer (or computer network). Thus, the assessment in step (c) is carried out by a processing unit. Preferably, said assessment is based on the results of step (b).

The value or values received in step (a) shall be derived from the determination of the amount of the biomarker from a subject as described elsewhere herein. Preferably, the value is a value for the concentration of the biomarker. The value will be typically received by the processing unit by uploading or sending the value to the processing unit. Alternatively, the value can be received by the processing unit by inputting the value via an user interface.

In an embodiment of the aforementioned method, the reference (or references) set forth in step (b) is (are) established from a memory. Preferably, a value for the reference is established from the memory.

In an embodiment of the aforementioned computer-implemented method of the present invention, the result of the assessment made in step c) is provided via a display, configured for presenting result.

In an embodiment of the aforementioned computer-implemented method of the present invention, the method may comprise the further step of transferring the information on the assessment made in step c) to the subject's electronic medical records.

Moreover, the present invention relates to the use (in particular, the in vitro use, e.g. in a sample from a subject) of

i) the biomarker TFPI-2 and optionally of at least one further biomarker selected from the group consisting of a natriuretic peptide, ESM-1 (Endocan), Ang2 and IGFBP7 (Insulin-like growth factor-binding protein 7), and/or

ii) at least one agent that specifically binds to TFPI-2, and, optionally, at least one further agent selected from the group consisting of an agent which specifically binds to a natriuretic peptide, an agent which specifically binds to ESM-1, an agent which specifically binds to Ang2 and an agent which specifically binds to IGFBP7,

for a) assessing atrial fibrillation, b) predicting the risk of stroke in a subject, c) improving the prediction accuracy of a clinical stroke risk score, for assessing the efficacy of an anticoagulation therapy of a subject, d) for identifying a subject being eligible for the administration of at least one anticoagulation medicament or being eligible for intensifying anticoagulation therapy (or reducing anticoagulation therapy e.g. by reducing the dosage of at least one anticoagulation medicament), and/or e) monitoring anticoagulation therapy.

In particular, the present invention relates to the use (in particular the in vitro use, e.g. in a sample of a subject) of

i) the biomarker TFPI-2 and optionally of at least one further biomarker selected from the group consisting of a natriuretic peptide, ESM-1 (Endocan), Ang2 and IGFBP7 (Insulin-like growth factor-binding protein 7), and/or

ii) at least one agent that specifically binds to TFPI-2, and, optionally, at least one further agent selected from the group consisting of an agent which specifically binds to a natriuretic peptide, an agent which specifically binds to ESM-1, an agent which specifically binds to Ang2 and an agent which specifically binds to IGFBP7, for predicting stroke.

The present invention further concern the use of
i) the biomarker TFPI-2 and optionally of at least one further biomarker selected from the group consisting of a natriuretic peptide, ESM-1 (Endocan), Ang2 and IGFBP7 (Insulin-like growth factor-binding protein 7), and/or
ii) at least one agent that specifically binds to TFPI-2, and, optionally, at least one further agent selected from the group consisting of an agent which specifically binds to a natriuretic peptide, an agent which specifically binds to ESM-1, an agent which specifically binds to Ang2 and an agent which specifically binds to IGFBP7, in a sample from a subject, in combination with a clinical stroke risk score, for predicting the risk of a subject to suffer from stroke.

Also, the present invention relates to the use (in particular the in vitro use, e.g. in a sample of a subject) of

i) the biomarker TFPI-2 and optionally of at least one further biomarker selected from the group consisting of a natriuretic peptide, ESM-1 (Endocan), Ang2 and IGFBP7 (Insulin-like growth factor-binding protein 7), and/or

ii) at least one agent that specifically binds to TFPI-2, and, optionally, at least one further agent selected from the group consisting of an agent which specifically binds to a natriuretic peptide, an agent which specifically binds to ESM-1, an agent which specifically binds to Ang2 and an agent which specifically binds to IGFBP7,

for improving the prediction accuracy of a clinical stroke risk score.

The terms mentioned in connection with the aforementioned use such as “sample”, “subject”, “detection agent”, “specifically binding”, “atrial fibrillation”, and “assessing atrial fibrillation” have been defined in connection with the method for assessing atrial fibrillation. The definitions and explanations apply accordingly.

Preferably, the aforementioned use is an in vitro use. Moreover, the detection agent is preferably and antibody such as a monoclonal antibody (or an antigen binding fragment thereof).

The present invention also relates to a kit. In an embodiment, the kit of the present invention comprises an agent which specifically binds to TFPI-2 and at least one further agent selected from the group consisting of an agent which specifically binds to a natriuretic peptide, an agent which specifically binds to ESM-1, an agent which specifically binds Ang2 and an agent which specifically binds to IGFBP7.

Preferably, said kit is adapted for carrying out the method of the present invention, i.e. the method for assessing atrial fibrillation. Optionally, said kit comprises instructions for carrying out the said method.

The term “kit” as used herein refers to a collection of the aforementioned components, preferably, provided separately or within a single container. The container also comprises instructions for carrying out the method of the present invention. These instructions may be in the form of a manual or may be provided by a computer program code which is capable of carrying out the calculations and comparisons referred to in the methods of the present invention and to establish the assessment or diagnosis accordingly when implemented on a computer or a data processing device. The computer program code may be provided on a data storage medium or device such as an optical storage medium (e.g., a Compact Disc) or directly on a computer or data processing device. Moreover, the kit may, preferably, comprise standard amounts for the biomarker TFPI-2 for calibration purposes. In a preferred embodiment, the kit further comprises standard amounts for the natriuretic peptide for calibration purposes

In an embodiment, said kit is used for assessing atrial fibrillation in vitro.

The figures show:

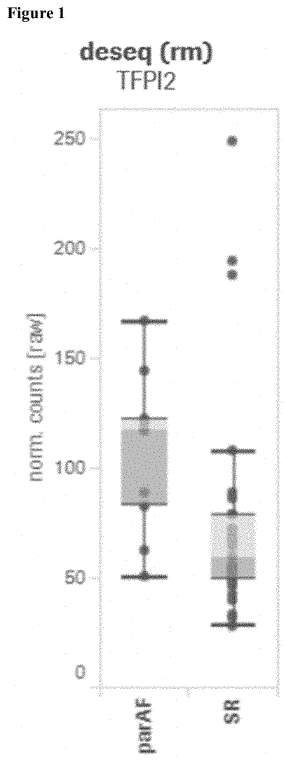

FIG. 1: Measurement of TFPI-2 in Mapping study: Exploratory AFib panel: Patients with a history of atrial fibrillation undergoing open chest surgery and epicardial mapping of paroxysmal AF, persistent AF or SR (Mapping study). Atrial tissue RNA expression profiles were assessed.

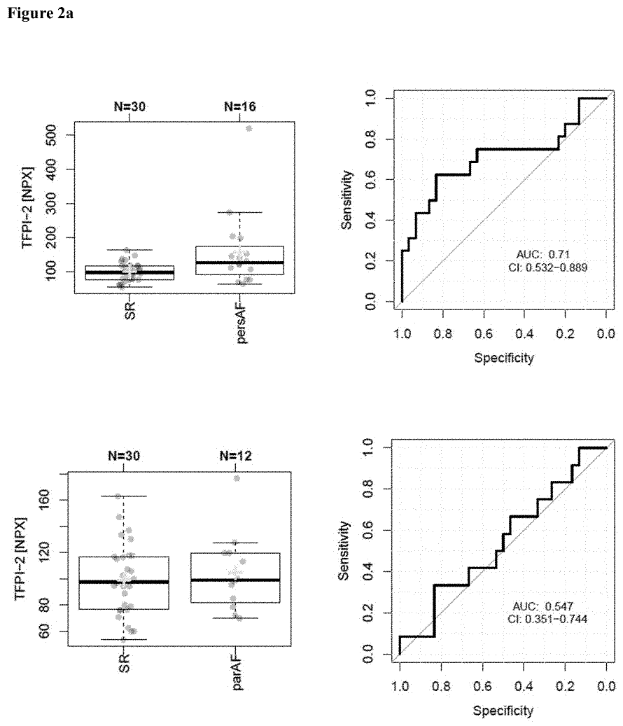

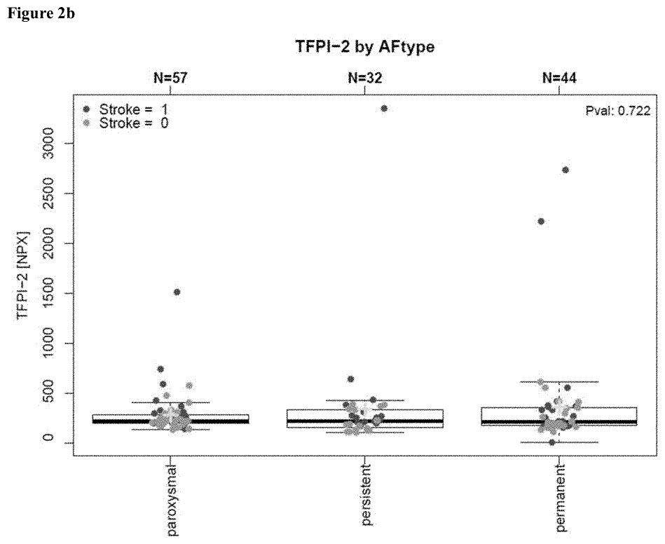

FIG. 2a: Measurement of TFPI-2 in Mapping study: Exploratory AFib panel: Patients with a history of atrial fibrillation undergoing open chest surgery and epicardial mapping of paroxysmal AF, persistent AF or SR (Mapping study). Circulating TFPI-2 levels were assessed.

FIG. 2b: Measurement of TFPI-2 in Beat AF study: AFib panel with stroke outcome: Patients with different types of atrial fibrillation paroxysmal AF, persistent AF and permanent AF. Circulating TFPI-2 levels were assessed.

FIG. 3: Prediction the risk of stroke TFPI-2 (Beat AF study): The FIG. 3 shows, that elevated titers of TFPI-2 associate to increased risk of stroke. TFPI-2 improved the C-Index of several clinical risk scores.

FIG. 4: Correlation to NTproBNP and ESM-1 in Beat AF: FIG. 4 shows that TFPI-2 has only low correlation with established markers (NTproBNP and ChadsVasc) as well as with ESM-1: a) TFPI-2 vs NTproBNP correlation coefficient=0.22, b) TFPI-2 vs ESM1 correlation coefficient=0.32 c) TFPI-2 vs CHADsVASc. correlation coefficient=0.13. These data suggest, that TFPI-2 provides complementary information and combinations of TFPI-2 and/or NTproBNP and/or ESM1 and/or CHADsVASc markers may provide improved detection of patients at high risk of stroke vs each marker alone.

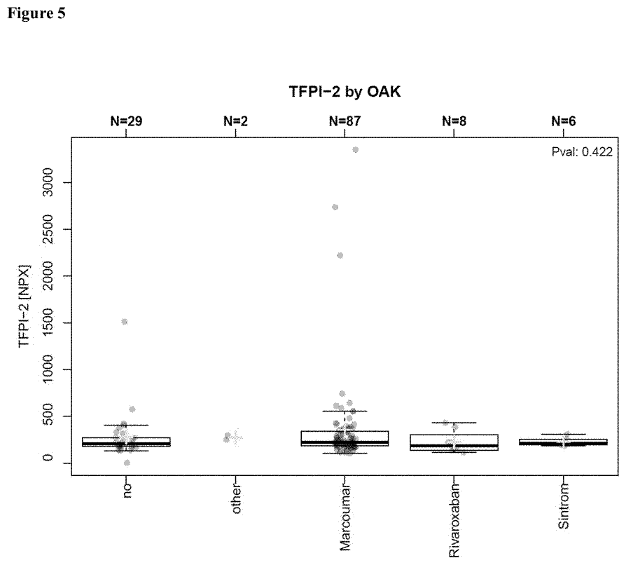

FIG. 5: TFPI-2 values observed in the BEAT-AF study separated by intake of oral anticoagulation: Patients which use Rivaroxaban show lower concentrations of TFPI-2 compared to the remaining patients.

### EXAMPLES

The invention will be merely illustrated by the following Examples. The said Examples shall, whatsoever, not be construed in a manner limiting the scope of the invention.

### Example 1: Differential Expression of TFPI-2 in Cardiac Tissue of AF Patients

Differential TFPI-2 expression levels have been determined in myocardial tissue samples from the right atrial appendage of n=38 patients.

RNAseq Analyses

Atrial tissue was sampled during open chest surgery because of CABG or valve surgery. Evidence of AF or SR (controls) was generated during surgery with simultaneous EndoEpicardial High Density Activation Mapping. Patients with AF and controls were matched with regard to gender, age and comorbidities.

Atrial tissue samples were prepared for
AF patients; n=9 patients
control patients in SR; n=29 patients.

Differential expression of TFPI-2 was determined in RNAseq analyses applying the algorithms RSEM and DESEQ2.

As shown in FIG. 1, TFPI-2 expression was found to be upregulated in the analyzed atrial tissues of the 9 patients with paroxysmal AF versus the 29 control patients.

The fold change in expression (FC) was 1.221.

The altered expression of TFPI-2 was determined in the damaged end organ, the atrial tissue. TFPI-2 mRNA levels were compared to results of high density mapping of the atrial tissue. Elevated TFPI-2 mRNA levels were detected in atrial tissue samples with conduction disturbances as characterized by electrical mapping. Conductance disturbances may be caused by fat infiltration or by interstitial fibrosis. The observed differential expression of TFPI-2 in atrial tissue of patients suffering from atrial fibrillation supports, that TFPI-2 is released in the circulation from the myocardium, in particular from the right atrial appendage and elevated serum/plasma titers assist the detection of episodes of AF.

It is concluded, that TFPI-2 is released from the heart into the blood and may aid the detection of AF episodes and predict the risk of developing AF related stroke.

### Example 2: Assessment of AF with Circulating TFPI-2

The MAPPING study related to patients undergoing open chest surgery. Samples were obtained before anesthesia and surgery. Patients were electrophysiologically characterized using high-density epicardial mapping with multi-electrode arrays (high density mapping). Circulating TFPI-2 levels have been determined in 12 patients with paroxysmal atrial fibrillation, 16 patients with persistent atrial fibrillation and 30 controls, matched to best possible (on age, gender, comorbidities). TFPI-2 was determined in samples of the MAPPING study.

Measurements were performed in 30 patients with sinus rhythm (SR), in 12 patients with paroxysmal arterial fibrillation (parAF) and in 16 persistent arterial fibrillation (persAF). FIG. 2a shows that TFPI-2 is significantly elevated in patients with persAF in comparison to patients in SR (AUC 0.71). Therefor TFPI-2 could be used for aid in diagnosis of persAF. Elevated TFPI-2 values would indicate a higher probability of persAF.

FIG. 2a shows that in patients of the MAPPING study with parAF compared to patients with SR TFPI-2 values are still higher in the AF group (AUC 0.55), but no longer significant.

The Beat AF study related to baseline samples of AF patients followed for stroke outcome for 6 years.

FIG. 2b shows that comparable circulating TFPI-2 titers were detected in the patients with different types of AF in patients of the Beat AF study. The TFPI-2 titers observed in the AF patients of the Beat AF study were elevated versus the patients of the Mapping study in SR.

### Example 3: Prediction of Stroke

Analysis Approach

The ability of circulating TFPI-2 to predict the risk for the occurrence of stroke was assessed in a prospective, multicentric registry of patients with documented atrial fibrillation (Conen D., Forum Med Suisse 2012; 12:860-862). TFPI-2 was measured using a stratified case cohost design as described in Borgan (2000).

For each of the 70 patients which experienced a stroke during follow up (“events”), 1 matched control was selected. Controls were matched based on the demographic and clinical information of age, sex, history of hypertension, atrial fibrillation type and history of heart failure (CHF history).

TFPI-2, NTproBNP, ESM-1, Ang-2, IGFBP-7 results were available for 67 patients with an event and 66 patients without an event.

TFPI-2 was measured using the Olink platform therefor no absolute concentration values are available and can be reported. Results will be reported on an arbitrary signal scale (NPX). NTproBNP, Ang-2 and IGFBP-7 were measured using the Elecsys platform and ESM-1 was measured using an Elisa microtiter plate.

In order to quantify the univariate prognostic value of TFPI-2 proportional hazard models were used with the outcome stroke.

The univariate prognostic performance of TFPI-2 was assessed by two different incorporations of the prognostic information given by TFPI-2.

The first proportional hazard model included TFPI-2 binarized at the median (221 NPX) and therefore comparing the risk of patients with TFPI-2 below or equal to the median versus patient with TFPI-2 above the median.

The second proportional hazard model included the original TFPI-2 levels but transformed to a log 2 scale. The log 2 transformation was performed in order to enable a better model calibration.

Because the estimates from a naïve proportional hazard model on the case control cohort would be biased (due to the altered proportion of cases to controls) a weighted proportional hazard model was used. Weights are based on the inverse probability for each patient to be selected for the case control cohort as described in Mark (2006). In order to get estimates for the absolute survival rates in the two groups based on the dichotomized baseline TFPI-2 measurement (<=221 NPX vs >221 NPX) a weighted version of the Kaplan-Meier plot was created as described in Mark (2006).

In order to assess if the prognostic value of TFPI-2 is independent from known clinical and demographic risk factors a weighted proportional cox model including in addition the variables age, sex, CHF history, history of hypertension, Stroke/TIA/Thromboembolism history, vascular disease history and diabetes history was calculated.

In order to assess the ability of TFPI-2 to improve existing risk scores for the prognosis of stroke the CHADS₂ the CHA₂DS₂-VASc and the ABC score were extended by TFPI-2 (log 2 transformed). Extension was done by creating a portioned hazard model including TFPI-2 and the respective risk score as independent variables. NTproBNP, ESM-1, Ang-2 and IGFBP-7 were assessed for the ability to improve the CHA₂DS₂-VASc score.

The c-indices of the CHADS₂, the CHA₂DS₂-VASc and ABC score were compared to the c-indices of these extended models. For the calculation of the c-index in the case-cohort setting a weighted version of the c-index was used as proposed in Ganna (2011).

Results

Table 1 shows the results of the two univariate weighted proportional hazard models including the binarized or the log 2 transformed TFPI-2. The association between the risk for experiencing a stroke with the baseline value of TFPI-2 is highly significant in both models. The hazard ration for the binarized TFPI-2 implies a 2.36-fold higher risk for a stroke in the patient group with baseline TFPI-2>=221 NPX versus the patient group with baseline TFPI2<221 NPX. The results of the proportional hazard model including TFPI-2 as log 2 transformed linear risk predictor suggest the log 2 transformed values TFPI-2 are proportional to the risk for experiencing a stroke. The hazard ratio of 2.52 can be interpreted in a way that a 2-fold increase of TFPI-2 is associated with 2.52 increase of risk for a stroke.

In this context it is interesting to note that TFPI-2 level correlate with the intake of certain oral anticoagulants (OAKs). FIG. 5 shows that patients which use Rivaroxaban show lower concentrations of TFPI-2 compared to the remaining patients. But there are also some patients with intake of Rivaroxaban which have CES values above 221 NPX. This could indicate that TFPI-2 could be used to monitor the effectiveness of OAK intake.

TABLE 1
Results result of the univariate weighted proportional hazard
model including the binarized and log2 transformed TFPI-2.

| | Hazard Ratio (HR) | 95%-CI HR | P-Value |
|---|---|---|---|
| TFPI-2 log2 | 2.5153 | 1.2558-5.0380 | 0.0093 |
| Baseline TFPI-2 >= 221 | 2.3612 | 1.2007-4.6433 | 0.0128 |
| NPX vs TFPI-2 < 221 | | | |
| NPX | | | |

Table 2 shows the results of a proportional hazard model including TFPI-2 (log 2 transformed) in the combination with clinical and demographic variables. It clearly shows that the prognostic effect of TFPI-2 stays stable if adjusting for the prognostic effect of relevant clinical and demographic variables.

TABLE 2
Multivariate proportional hazard model including TFPI-2
and relevant clinical and demographic variables.

| | Hazard Ratio (HR) | 95%-CI HR | P-Value |
|---|---|---|---|
| History hypertension | 1.3471 | 0.5923-3.0639 | 0.4773 |
| Age | 1.0272 | 0.9814-1.075 | 0.2488 |
| History | 1.9457 | 0.7698-4.9179 | 0.1594 |
| Stroke/TIA/embolism | | | |
| Sex = male | 1.2565 | 0.4547-3.4722 | 0.6598 |
| History CHF | 0.6667 | 0.2718-1.6355 | 0.3759 |
| History vascular | 1.4862 | 0.5658-3.9041 | 0.4214 |
| disease | | | |
| TFPI-2 (log2 | 2.7248 | 1.0987-6.7577 | 0.0305 |
| transformed) | | | |

Table 3 shows the results of the weighted proportional hazard model combining the CHADS₂ score with TFPI-2 (log 2 transformed). Also in this model TFPI-2 can add prognostic information to the CHADS₂ score.

TABLE 3
Weighted proportional hazard model combining the
CHADS₂ score with TFPI-2 (log2 transformed)

| | Hazard Ratio (HR) | 95%-CI HR | P-Value |
|---|---|---|---|
| CHADS₂ score | 1.4450 | 1.1286-1.8502 | 0.0035 |
| TFPI-2 (log2 | 2.5818 | 1.3275-5.0212 | 0.0052 |
| transformed) | | | |

Table 4 shows the results of the weighted proportional hazard model combining the CHA₂DS₂-VASc score with TFPI-2 (log 2 transformed). Also in this model TFPI-2 can add prognostic information to the CHA₂DS₂-VASc score.

TABLE 4
Weighted proportional hazard model combining the
CHA₂DS₂-VASc score with TFPI-2 (log2 transformed)

| | Hazard Ratio (HR) | 95%-CI HR | P-Value |
|---|---|---|---|
| CHA₂DS₂-VASc | 1.3950 | 1.0784-1.8046 | 0.0112 |
| score | | | |
| TFPI-2 (log2 | 2.2217 | 1.1047-4.4683 | 0.0251 |
| transformed) | | | |

Table 5 shows the results of the weighted proportional hazard model combining the ABC score with TFPI-2 (log 2 transformed). Also in this model TFPI-2 can add prognostic information to the risk score.

TABLE 5
Weighted proportional hazard model combining
the ABC score with TFPI-2 (log2 transformed)

| | Hazard Ratio (HR) | 95%-CI HR | P-Value |
|---|---|---|---|
| ABC score | 1.1209 | 1.0004-1.256 | 0.0493 |
| TFPI-2 (log2 | 2.1272 | 0.7548-2.1272 | 0.0413 |
| transformed) | | | |

Table 6 shows the estimated c-indexes of TFPI-2 alone, of the CHADS₂, the CHA₂DS₂-VASc, the ABC score and of the weighted proportional hazard model combining the CHADS₂, the CHA₂DS₂-VASc, the ABC score with TFPI-2 (log 2) on the case cohort selection. It can be seen that the addition of TFPI-2 improves the c-index of all three risk models. The improvements are 0.0646, 0.0432 and 0.0671 for the CHADS₂, the CHA₂DS₂-VASc, the ABC score respectively.

Table 6 shows the estimated c-indexes of NTproBNP alone, of ESM-1 alone, of Ang-2 alone, of IGFBP-7 alone, of the CHA₂DS₂-VASc score and of the weighted proportional hazard model combining the CHA₂DS₂-VASc score with NTproBNP (log 2), with ESM-1 (log 2), with ANG-2 (log 2), with IGFBP-7 (log 2) on the case cohort selection. It can be seen that the addition of all biomarkers improve the c-index of the CHA₂DS₂-VASc score. The improvements of the the CHA₂DS₂-VASc score are 0.002, 0.064, 0.036 and 0.006 for NTproBNP, ESM-1, Ang-2, IGFBP-7 respectively.

TABLE 6
C-indexes of TFPI-2, NTproBNP, ESM-1, Ang-2, IGFBP-7, the
CHA₂DS₂-VASc score and the combination of the
CHA₂DS₂-VASc score with TFPI-2, NTproBNP,
ESM-1, Ang-2, IGFBP-7 and C-indexes of the CHADS₂ and
ABC score and their combination with TFPI-2.

| | C-Index |
|---|---|
| TFPI-2 univariate | 0.682 |
| CHADS₂ | 0.650 |
| CHADS₂ + TFPI-2 | 0.715 |
| CHA₂DS₂-VASc | 0.674 |
| CHA₂DS₂-VASc + TFPI-2 | 0.717 |
| ABC score | 0.648 |
| ABC score + TFPI-2 | 0.716 |
| NTproBNP univariate | 0.651 |
| CHA₂DS₂-VASc + NTproBNP | 0.676 |
| ESM-1 univariate | 0.708 |
| CHA₂DS₂-VASc + ESM-1 | 0.738 |
| Ang-2 univariate | 0.696 |
| CHA₂DS₂-VASc + Ang-2 | 0.710 |
| IGFBP-7 univariate | 0.652 |
| CHA₂DS₂-VASc + IGFBP-7 | 0.680 |

### Example 4: Biomarker Measurements

TFPI-2 was measured in a commercially available 0-link multi-marker panel for Tissue factor inhibitor-2 (TFPI-2); Proximity Extension Assay from O-link, Sweden.

### Example 5: Prediction of Stroke

Analysis Approach

The ability of circulating TFPI-2 to predict the risk for the occurrence of stroke was verified (in reference to example 3) in a prospective, multicentric registry of patients with documented atrial fibrillation (Conen D., Swiss Med Wkly. 2017 Jul. 10; 147:w14467). TFPI-2 was measured using a stratified case cohort design as described in Borgan (2000).

For each of the 68 patients (status April 2019) which experienced a stroke during follow up (“events”), 1 matched control was randomly selected out of 2319 patients without an event.

TFPI-2, results were available for 62 patients with an event and 61 patients without an event.

TFPI-2 was measured using the Olink platform therefor no absolute concentration values are available and can be reported. Results will be reported on an arbitrary signal scale (NPX). Please note that therefor no comparison are possible to example 3 in terms of concentration and distribution of the biomarker (e.g. median).

In order to quantify the univariate prognostic value of TFPI-2 proportional hazard models were used with the outcome stroke.

The univariate prognostic performance of TFPI-2 was assessed by two different incorporations of the prognostic information given by TFPI-2.

The first proportional hazard model included TFPI-2 binarized at the median (748 NPX) and therefore comparing the risk of patients with TFPI-2 below or equal to the median versus patient with TFPI-2 above the median.

The second proportional hazard model included the original TFPI-2 levels but transformed to a log 2 scale. The log 2 transformation was performed in order to enable a better model calibration.

Because the estimates from a naïve proportional hazard model on the case control cohort would be biased (due to the altered proportion of cases to controls) a weighted proportional hazard model was used. Weights are based on the inverse probability for each patient to be selected for the case control cohort as described in Mark (2006).

In order to get estimates for the absolute survival rates in the two groups based on the dichotomized baseline TFPI-2 measurement (<=748 NPX vs >748 NPX) a weighted version of the Kaplan-Meier plot was created as described in Mark (2006).

In order to assess if the prognostic value of TFPI-2 is independent from known clinical and demographic risk factors a weighted proportional cox model including in addition the variables age, and history of Stroke/TIA/Thromboembolism was calculated. These were the only significant clinical risk predictors on the whole cohort (including all controls).

In order to assess the ability of TFPI-2 to improve existing risk scores for the prognosis of stroke the CHADS₂ the CHA₂DS₂-VASc and the ABC score were extended by TFPI-2 (log 2 transformed). Extension was done by creating a portioned hazard model including TFPI-2 and the respective risk score as independent variables.

The c-indices of the CHADS₂, the CHA₂DS₂-VASc and ABC score were compared to the c-indices of these extended models. For the calculation of the c-index in the case-cohort setting a weighted version of the c-index was used as proposed in Ganna (2011).

Results

Table 7 shows the results of the two univariate weighted proportional hazard models including the binarized or the log 2 transformed TFPI-2. The association between the risk for experiencing a stroke with the baseline value of TFPI-2 is highly significant in both models. The hazard ration for the binarized TFPI-2 implies a 2.04-fold higher risk for a stroke in the patient group with baseline TFPI-2>=748 NPX versus the patient group with baseline TFPI2<748 NPX. The results of the proportional hazard model including TFPI-2 as log 2 transformed linear risk predictor suggest the log 2 transformed values TFPI-2 are proportional to the risk for experiencing a stroke. The hazard ratio of 2.54 can be interpreted in a way that a 2-fold increase of TFPI-2 is associated with 2.54 increase of risk for a stroke.

TABLE 7
Results result of the univariate weighted proportional hazard
model including the binarized and log2 transformed TFPI-2.

| | Hazard Ratio (HR) | 95%-CI HR | P-Value |
|---|---|---|---|
| TFPI-2 log2 | 2.5445 | 1.2078-5.3606 | 0.0140 |
| Baseline TFPI-2 >= 748 | 2.0365 | 0.9639-4.3030 | 0.0624 |
| NPX vs TFPI-2 < 748 | | | |
| NPX | | | |

Table 8 shows the results of a proportional hazard model including TFPI-2 (log 2 transformed) in the combination with clinical and demographic variables. It clearly shows that the prognostic effect of TFPI-2 stays stable if adjusting for the prognostic effect of relevant clinical and demographic variables.

TABLE 8
Multivariate proportional hazard model including TFPI-2
and relevant clinical and demographic variables.

| | Hazard Ratio (HR) | 95%-CI HR | P-Value |
|---|---|---|---|
| Age | 1.1347 | 1.0632-1.2110 | 0.2488 |
| History | 6.7040 | 2.5049-17.9425 | 0.1594 |
| Stroke/TIA/embolism | | | |
| TFPI-2 (log2 | 2.7248 | 1.1610-6.7540 | 0.0305 |
| transformed) | | | |

Table 9 shows the results of the weighted proportional hazard model combining the CHADS₂ score with TFPI-2 (log 2 transformed). Also in this model TFPI-2 can add prognostic information to the CHADS₂ score.

TABLE 9
Weighted proportional hazard model combining the
CHADS₂ score with TFPI-2 (log2 transformed)

| | Hazard Ratio (HR) | 95%-CI HR | P-Value |
|---|---|---|---|
| CHADS₂ score | 1.4175 | 0.9690-2.0737 | 0.0722 |
| TFPI-2 (log2 | 2.7709 | 1.2504-6.1405 | 0.0121 |
| transformed) | | | |

Table 10 shows the results of the weighted proportional hazard model combining the CHA₂DS₂-VASc score with TFPI-2 (log 2 transformed). Also in this model TFPI-2 can add prognostic information to the CHA₂DS₂-VASc score.

TABLE 10
Weighted proportional hazard model combining the
CHA₂DS₂-VASc score with TFPI-2 (log2 transformed)

| | Hazard Ratio (HR) | 95%-CI HR | P-Value |
|---|---|---|---|
| CHA₂DS₂-VASc | 1.3497 | 0.9496-1.9185 | 0.0946 |
| score | | | |
| TFPI-2 (log2 | 2.4365 | 1.1391-5.2114 | 0.0217 |
| transformed) | | | |

Table 11 shows the results of the weighted proportional hazard model combining the ABC score with TFPI-2 (log 2 transformed). Also in this model TFPI-2 can add prognostic information to the risk score.

TABLE 11
Weighted proportional hazard model combining
the ABC score with TFPI-2 (log2 transformed)

| | Hazard Ratio (HR) | 95%-CI HR | P-Value |
|---|---|---|---|
| ABC score | 1.2692 | 1.0818-1.4892 | 0.0035 |
| TFPI-2 (log2 | 1.9093 | 0.8658-4.2106 | 0.1090 |
| transformed) | | | |

Table 12 shows the estimated c-indexes of TFPI-2 alone, of the CHADS₂, the CHA₂DS₂-VASc, the ABC score and of the weighted proportional hazard model combining the CHADS₂, the CHA₂DS₂-VASc, the ABC score with TFPI-2 (log 2) on the case cohort selection. It can be seen that the addition of TFPI-2 improves the c-index of all three risk models. The improvements are 0.057, 0.047 and 0.022 for the CHADS₂, the CHA₂DS₂-VASc, the ABC score respectively.

TABLE 12
C-indexes of TFPI-2, the CHA₂DS₂-VASc score and the combination
of the CHA₂DS₂-VASc score with TFPI-2 and C-indexes of the
CHADS₂ and ABC score and their combination with TFPI-2.

| | C-Index |
|---|---|
| TFPI-2 univariate | 0.608 |
| CHADS₂ | 0.573 |
| CHADS₂ + TFPI-2 | 0.630 |
| CHA₂DS₂-VASc | 0.577 |
| CHA₂DS₂-VASc + TFPI-2 | 0.624 |
| ABC score | 0.680 |
| ABC score + TFPI-2 | 0.702 |

### Example 6: Case Studies

There is growing interest in knowing and reducing the ischemic stroke risk also in patients without atrial fibrillation (Yao X et al, Am Heart J. 2018; 199:137-143). For example, predicting the stroke risk is essential to establish optimum treatment strategies by identifying and including these patients at high stroke risk into drug studies with oral anticoagulation.

The CHA2DS2-VASc score, for example, predicts incidence of ischemic stroke also in patients without atrial fibrillation, but with a lower absolute event rate (Mitchell L B et al, Heart. 2014; 100:1524-30). (Therefore, it is less clear, if and at what CHA2DS2-VASc score these patients without atrial fibrillation should receive oral anticoagulation (OAC) and at which dose, so that biomarkers such as TFPI2 help to assess the need for therapy and effectiveness of OAC.

A 76 year old female patient with hypertension and no history of atrial fibrillation presents in sinus rhythm. TFPI2 is determined in an EDTA plasma sample obtained from the patient. The clinical information of the CHA2DS2-VASc score (advanced age and hypertension) indicate a certain stroke risk, and in addition the TFPI2 value is above a reference value. The elevated TFPI2 titers is indicative of high risk to experience a stroke. As consequence the patient is admitted to an anticoagulation therapy.

A 65 year old male patient without a history of atrial fibrillation requests a checkup at the doctor's office. The presents in sinus rhythm, however structural heart disease is diagnosed. Because of the history of stroke and high overall CHA2DS2-VASc score, the patient already receives oral anticoagulation therapy (at low dose). In order to determine the current risk of stroke and to conclude on eventual therapy change, TFPI2 is measured in a serum sample obtained from the patient. The observed TFPI2 value is above a reference value. The elevated TFPI2 titers and other risk parameters (history of stroke) are indicative of a high residual stroke risk that is higher than the bleeding risk (assessed with other clinical information). As consequence the dosage of the anticoagulation therapy is increased.

A 68 year old obese female patient with Diabetes Mellitus and heart failure with reduced ejection fraction presents with acute symptoms of shortness of breath. In prior visits, the patient has no history of atrial fibrillation. According to a high overall CHA2DS2-VASc risk score, the physician decided to start oral anticoagulation (low dose) even in the absence of AFib. The TFPI2 level was determined before and after onset of anticoagulation. The patient now is wondering whether the anticoagulation therapy is effective and still necessary. In order to specify the current risk of stroke, TFPI2 is determined in an EDTA sample obtained from the patient. The observed TFPI2 value is below a reference value and lower as compared to the treatment start. The reduced TFPI2 titers are indicative of an effective anticoagulation therapy. As consequence the anticoagulation therapy is maintained.

Summary: The studies underlying the present invention show that TFPI-2 can be used to diagnose AF, to classify AF, to assess the AF severity, to guide therapy (with objectives to therapy intensification/reduction), to predict disease outcome (risk prediction, e.g. stroke), therapy monitoring, therapy stratification (selection of therapy options).

1. A method for assessing atrial fibrillation in a subject, comprising the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby atrial fibrillation is to be assessed.

2. The method of claim 1, wherein the sample is a blood, serum or plasma sample.

3. The method according to claim 1, wherein an amount of TFPI-2 and, optionally, an amount of the at least one further biomarker above the reference amount is indicative for a subject suffering from atrial fibrillation and/or wherein an amount of TFPI-2 and, optionally, an amount of the at least one further biomarker below the reference amount is indicative for a subject not suffering from atrial fibrillation,
or
wherein the subject is suffering from atrial fibrillation, and wherein an amount of TFPI-2 and, optionally, an amount of the at least one further biomarker above the reference amount is indicative for a subject suffering from persistent atrial fibrillation and/or wherein an amount of TFPI-2 and, optionally, an amount of the at least one further biomarker below the reference amount is indicative for a subject suffering from paroxysmal atrial fibrillation.

4. The method of claim 1, wherein the assessment of atrial fibrillation is the prediction of the risk of stroke associated with atrial fibrillation, and
wherein an amount of TFPI-2 and, optionally, an amount of the at least one further biomarker above the reference amount is indicative for a subject who is at risk of suffering from stroke associated with atrial fibrillation and/or wherein an amount of TFPI-2 and, optionally, an amount of the at least one further biomarker below the reference amount is indicative for a subject who is not at risk of suffering from stroke associated with atrial fibrillation.

5. A method for predicting the risk of stroke in a subject, comprising the steps of
(a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
(b) assessing the clinical stroke risk score for said subject, and
(c) predicting the risk of stroke based on the results of steps a) and b).

6. A method for assessing the efficacy of an anticoagulation therapy of a subject, comprising the steps of
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
a) comparing the amount of the biomarker TFPI-2 to a reference amount for TFPI-2 and, optionally, comparing the amount of the at least one further biomarker to a reference amount for said at least one further biomarker, whereby the efficacy of the anticoagulation therapy is to be assessed.

7. A method for identifying a subject being eligible to the administration of at least one anticoagulation medicament or being eligible to an intensified anticoagulation therapy, comprising
a) determining, in at least one sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), and
b) comparing the amount of the biomarker TFPI-2 to a reference amount for TFPI-2 and, optionally, comparing the amount of the at least one further biomarker to a reference amount for said at least one further biomarker, whereby a subject being eligible to the administration of said at least one medicament or to an intensified anticoagulation therapy is identified.

8. The method of claim 7, wherein a subject who is eligible to an intensified anticoagulation therapy is eligible to an increase of the dosage of the currently administered anticoagulant, or to the replacement of a currently administered anticoagulant with a more effective anticoagulant.

9. A method for monitoring anticoagulation therapy, comprising the steps of
(a) determining, in at first sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7),
(b) determining, in a second sample from the subject, the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 (Angiopoietin 2) and IGFBP7 (Insulin-like growth factor-binding protein 7), wherein said second sample has been obtained after said first sample,
(c) comparing the amount of TFPI-2 in the first sample to the amount of TFPI-2 in said second sample, and optionally comparing the amount of said at least one further biomarker in the first sample to the amount of said at least one further biomarker in said second sample, thereby monitoring anticoagulation therapy.

10. The method according to claim 5, wherein the subject suffers from atrial fibrillation.

11. A method of aiding in the assessment of atrial fibrillation, said method comprising the steps of:
a) providing at least one sample from a subject,
b) determining, in the at least one sample provided in step a), the amount of the biomarker TFPI-2 (Tissue Factor Pathway Inhibitor 2) and, optionally, the amount of at least one further biomarker selected from the group consisting of a natriuretic peptide, ESM-1 (Endocan), Ang2 and IGFBP7 (Insulin-like growth factor-binding protein 7), and
c) providing information on the determined amount of the biomarker TFPI-2 and optionally on the determined amount of the at least one further biomarker to a physician, thereby aiding in the assessment of atrial fibrillation.

12. A method for aiding in the assessment of atrial fibrillation, comprising:
a) providing an assay for the biomarker TFPI-2 and, optionally, at least one further assay for a further biomarker selected from the group consisting of a natriuretic peptide, ESM-1 (Endocan), Ang2 and IGFBP7 (Insulin-like growth factor-binding protein 7), and
b) providing instructions for using of assay results obtained or obtainable by said assay(s) in the assessment of atrial fibrillation.

13. A computer-implemented method for assessing atrial fibrillation, comprising
a) receiving, at a processing unit, a value for the amount of TFPI-2, and, optionally at least one further value for the amount of at least one further biomarker selected from the group consisting of a natriuretic peptide, ESM-1 (Endocan), Ang2 and IGFBP7 (Insulin-like growth factor-binding protein 7), wherein said amount of TFPI-2 and, optionally, the amount of the at least one further biomarker have been determined in a sample from a subject,
b) comparing, by said processing unit, the value or values received in step (a) to a reference or to references, and
c) assessing atrial fibrillation based in the comparison step b).

14. A kit comprising an agent which specifically binds to TFPI-2 and at least one further agent selected from the group consisting an agent which specifically binds to a natriuretic peptide, an agent which specifically binds to ESM-1, an agent which specifically binds Ang2 and an agent which specifically binds to IGFBP7.

15. In vitro use of
i) the biomarker TFPI-2 and optionally of at least one further biomarker selected from the group consisting of a natriuretic peptide, ESM-1 (Endocan), Ang2 and IGFBP7 (Insulin-like growth factor-binding protein 7), and/or
ii) at least one agent that specifically binds to TFPI-2, and, optionally, at least one further agent selected from the group consisting an agent which specifically binds to a natriuretic peptide, an agent which specifically binds to ESM-1, an agent which specifically binds to Ang2 and an agent which specifically binds to IGFBP7,
for a) assessing atrial fibrillation, b) predicting the risk of stroke in a subject, c) improving the prediction accuracy of a clinical stroke risk score, for assessing the efficacy of an anticoagulation therapy of a subject, d) for identifying a subject being eligible to the administration of at least one anticoagulation medicament or being eligible to an intensified anticoagulation therapy, and/or e) monitoring anticoagulation therapy.

16. The method of claim 7, wherein the replacement of the currently administered anticoagulant with a more effective anticoagulant comprises the replacement of a currently administered vitamin K antagonist with an oral anticoagulant

17. The method of claim 16, wherein the vitamin K antagonist is selected from the group consisting of warfarin and dicumarol.

18. The method of claim 16, wherein the more effective anticoagulant is selected from the group consisting of dabigatran, rivaroxaban and apixaban.
